(19) United States
(12) Patent Application Publication
(10) Pub. No.: US 2024/0301230 A1
(43) Pub. Date: Sep. 12, 2024

(54) BACTERIAL CELLULOSE SUSPENSIONS

(71) Applicant: CELLUGY APS, Soborg (DK)

(72) Inventors: Isabel Alvarez-Martos, Brabrand (DK); Deby Fapyane, Aarhus C (DK); Alixander Perzon, Malmö (SE)

(21) Appl. No.: 18/552,039

(22) PCT Filed: Mar. 28, 2022
(86) PCT No.: PCT/EP2022/058128

§ 371 Date: Sep. 22, 2023

(30) Foreign Application Priority Data
Mar. 26, 2021 (EP) 21165137.7

(51) Int. Cl. C09D 101/02; C08K 3/36; C08L 1/02; C09D 7/40; C09D 7/65; C12P 19/04; D21H 19/12; D21H 19/40; D21H 19/42; D21H 19/52; D21H 19/60

(52) U.S. Cl. CPC C09D 101/02; C08L 1/02; C09D 7/40; C09D 7/65; C09D 7/66; C12P 19/04; D21H 19/12; D21H 19/40; D21H 19/42; D21H 19/52; D21H 19/60; C08K 3/36; C08K 2201/006

(57) ABSTRACT

The invention presented herein relates to bacterial cellulose (BC) suspensions, and methods of production and uses of the same. In particular, the invention relates to BC suspensions suitable for coating applications.

### TECHNICAL FIELD

The present invention relates to bacterial cellulose suspensions, and methods of production and uses of the same.

### BACKGROUND

From 1950 to today, over 8.3B tonnes of plastic have been produced, and the demand for plastics has outpaced all other bulk materials such as steel, aluminium and cement. Plastic is everywhere around us: from packaging to textiles, building and construction, consumer goods, transportation and other uses such as in personal and/or home care. This is no surprise, as plastic is both versatile and cheap to produce. Its valuable properties include durability, high mechanical strength, and resistance to chemicals, fire and weather. Furthermore, its appearance can be customised in infinite ways. On top of this, plastic is so cheap to produce from fossil sources that it is easier to produce new plastic than to recycle existing plastic. Of all the plastic produced, 44% has been manufactured since 2000, and the amount is projected to increase by 40% by 2030, reaching 12B tonnes (WWF, 2019). Packaging alone represents 40% of global plastic production, and it is a necessary part of the food chain: packaging extends food life by 75%, allowing for long distance transportation and distribution (Gray, 2018).

There are several types of plastics being used as coating or barrier material for paper or other materials. Mostly, plastics such as poly vinyl chloride (PVDC) and ethylene vinyl alcohol (EVOH) serve only as moisture or oxygen barrier, thus they are used in combination to achieve both properties. PVDC is a fossil-based and non-biodegradable material consisting of 85% vinylidene chloride and 15% vinyl chloride (Solvay, Diofan®) (Diofan® technical sheet). It has a low oxygen transmission rate (OTR) and water vapor transmission rate (WVTR). EVOH is a fossil-based and non-biodegradable plastic and is composed of 24-48% ethylene and vinyl alcohol. EVOH provides better oxygen barrier properties than PVDC when dry, i.e. 0% relative humidity. However, a significant issue concerning the use of EVOH as a barrier material is its moisture sensitivity (EVAL® technical sheet).

Bioplastics, derived from bio-based sources, such as polylactic acid/polylactide (PLA) and polyhydroxyalkanoate (PHA), are currently the best option to unsustainable, conventional plastics. The two main characteristics of bioplastics include that they are bio-based (i.e. not made from fossil sources) and biodegradable (i.e. may be decomposed by microorganisms). Nowadays, bioplastics represent roughly 1% of the 335 M tonnes of plastic produced annually. A series of drawbacks prevents bioplastics from replacing conventional fossil-based plastic in several settings such as the packaging industry. These drawbacks include bioplastics being land intensive, unrecyclable, and, unfit for market due to lack of performance compared to conventional plastics. Although PLA is used in specialty papers to enhance their barrier properties, it generally demands a high coat weight or even double coating to achieve the necessary properties. Moreover, it displays poor compatibility with natural fibers when used as a reinforcing agent. Therefore, there are still further needs to improve the properties of PLA for its use as a barrier or additive in specialty papers. PHA has a poor melt flow stability, severely limiting its processing capabilities and range of applications. It is usually blended with other polymers or plasticizers, such as EVOH or PLA, to improve its processability and mechanical properties (van den Oever).

Cellulose is one of the most abundant, renewable and widely used natural polymers and is present in a variety of plant sources. Nanocellulose is a sustainable material which can replace plastics used for oxygen barrier coating. In general, the properties of plant nanocellulose changes depending on the weather and other conditions that influence the growth of the plant, which possesses a challenge for its use. The composition of any biopolymer attached to the nanocellulose moiety will heavily affect the plant nanocellulose's resistance towards salt or any other additives. Cellulose can also be obtained from microorganisms, such as certain fungi, algae, and bacteria. The cellulose produced by bacteria is commonly referred to as bacterial cellulose (BC), which is a sustainable option to cellulose derived from plants. BC comprises an ultra-fine and highly pure fiber network structure. While BC has the same molecular formula as plant cellulose, it has significantly different macromolecular properties and characteristics; in general, BC has a higher optical transparency and is more chemically pure, containing no hemicellulose or lignin. It also has a greater tensile strength and normally a higher water holding capacity and hydrophilicity than plant-derived cellulose. Additionally, BC has a more crystalline structure, and it forms thin microfibrils which are significantly smaller than those of plant cellulose (Choi & Shin, 2020).

BC is usually produced as pellicles (from static fermentation) or in dispersed form (from high shear agitation fermentation). In its pristine form, its application is very limited. Thus, formulation often include converting BC pellicles into suspension. Suspensions of BC can be prepared by comminution (disintegration) of the produced BC material. Such formulations may be useful in the pharmaceutical, cosmetics, food, chemical, and personal hygiene industry. BC suspensions can for example be used as emulsion stabilizers, thickeners, or as coating agents.

The use of BC suspensions for coating materials is a more sustainable option compared to coating with fossil-based plastic. Speciality papers, paper packaging or any fiber-based material such as cellulose-based textile coated with a BC suspension can be recycled into the respective recycling stream. Thus, it is effectively turning multi-material packaging into a mono-material which is beneficial both commercially and environmentally.

Hence, improved, bio-based and biodegradable BC suspensions suitable for coating of various materials, as well efficient processes for producing such suspensions, would be highly advantageous.

### SUMMARY

The project leading to this application has received funding from the European Union's Horizon 2020 research and innovation programme under grant agreement No 101010323.

The invention is as defined in the claims.

The invention presented herein relates to bacterial cellulose (BC) suspensions, and methods of production and uses of the same. In particular, the invention relates to BC suspensions suitable as particle or emulsion stabilizers, thickeners, and/or for coating applications of cellulose fiber-based material such as paper and textiles such as for creating a barrier against oxygen or other gasses, water and/or grease for various materials such as paper, cardboard, specialty paper, textile, cellulose- and/or fiber-based materials. The emulsification and/or particle stabilization properties of the BC suspensions of the invention is suitable for material coating applications as it enables and/or facilitates the addition of colorant(s) and/or oil(s) to the coating formula containing the BC suspension which might otherwise be challenging. Disclosed herein is also BC suspensions and methods of their production containing highly crystalline BC which contributes with higher water barrier capacity of a material upon coating with the BC suspension or formulations thereof compared to BC of lower crystallinity (crystallinity %<80%).

Thus, herein is provided a BC suspension comprising 1.6 to 2.3% (w/v) of BC solids, such as between 1.6 and 2.2% (w/v) of BC solids, such as between 1.6 and 2.1% (w/v) of BC solids, such as between 1.6 and 2.0% (w/v) of BC solids, such as between 1.6 and 1.9% (w/v) of BC solids, such as between 1.6 and 1.8% (w/v) of BC solids, such as between 1.6 and 1.7% (w/v) of BC solids, such as between 1.7 and 2.3% (w/v) of BC solids, such as 1.7 and 2.2% (w/v) of BC solids, such as between 1.7 and 2.1% (w/v) of BC solids, such as between 1.7 and 2.0% (w/v) of BC solids, such as between 1.7 and 1.9% (w/v) of BC solids, such as between 1.7 and 1.8% (w/v) of BC solids, such as between 1.8 and 2.3% (w/v) of BC solids, such as between 1.8 and 2.2% (w/v) of BC solids, such as between 1.8 and 2.1% (w/v) of BC solids, such as between 1.8 and 2.0% (w/v) of BC solids, such as between 1.8 and 1.9% (w/v) of BC solids, such as between 1.9 and 2.3% (w/v) of BC solids, such as between 1.9 and 2.2% (w/v) of BC solids, such as between 1.9 and 2.1% (w/v) of BC solids, such as between 1.9 and 2.0% (w/v) of BC solids. Preferably, said BC suspension comprises BC fiber clusters having a size between 10 nm and 1500 μm, and/or said BC suspension having a viscosity between 1 and 500 Pa·s at a shear stress of 1/s.

Also provided a BC suspension comprising 0.5 to 4% (w/v) of BC solids, such as between 0.5 and 1% (w/v) of BC solids, such as between 1 and 2% (w/v) of BC solids, such as between 2 and 3% (w/v) of BC solids, such as between 3 and 4% (w/v) of BC solids.

Further provided herein is a method for producing a BC suspension comprising the steps of:
a) Incubating at least one cellulose producing bacteria in a culture medium, and optionally, at least one other microorganism which does not produce cellulose, thereby obtaining BC in said culture medium;
b) Recovering said BC;
c) Optionally, contacting said BC with a functionalization agent, wherein said functionalization agent is selected from nitric acid and acetic anhydride, thereby functionalizing said BC;
d) Converting the recovered and/or the functionalized BC into a BC suspension comprising 0.5 to 4.0% (w/v) BC solids, such as between 0.5 and 1% (w/v) of BC solids, such as between 1 and 2% (w/v) of BC solids, such as between 2 and 3% (w/v) of BC solids, such as between 3 and 4% (w/v) of BC solids.

Also provided herein is a method for producing a BC suspension comprising the steps of:
a) Incubating at least one cellulose producing bacteria in a culture medium, and optionally, at least one other microorganism which does not produce cellulose, thereby obtaining BC in said culture medium;
b) Recovering said BC;
c) Optionally, contacting said BC with a functionalization agent, wherein said functionalization agent is selected from nitric acid and acetic anhydride, thereby functionalizing said BC;
d) Converting the recovered BC of step b) and/or the functionalized BC of step c) into a BC suspension comprising 1.6 to 2.3% (w/v) BC solids, such as between 1.6 and 2.2% (w/v) of BC solids, such as between 1.7 and 2.3% (w/v) of BC solids, such as between 1.8 and 2.3% (w/v) of BC solids, such as between 1.6 and 1.9% (w/v) of BC solids.

Preferably, said BC suspension comprising BC fiber clusters having a size between 10 nm and 1500 μm, and/or said BC suspension having a viscosity between 1 and 500 Pa·s at a shear stress of 1/s.

In some embodiments, the method further comprises a step of sterilisation, preferably said step is performed before steps a to d. Preferably, said step is performed before step c.

In some embodiments, the method further comprises further incubating at least one microorganism producing or being capable of producing at least one cellulytic agent in said culture medium.

Also provided is a BC suspension obtained from the methods described herein.

Also provided is a BC suspension, wherein said BC suspension is as defined herein.

Further provided herein is the use of the BC suspension for coating a material, such as the surface of a material.

Further provided herein is the use of the BC suspension as particle stabilizer (particle stabilizator) and/or emulsion stabilizer (emulsifier) and/or thickener.

Also provided is a method of producing a particle stabilizer (particle stabilizator) and/or emulsion stabilizer (emulsifier) and/or thickener comprising the BC suspension as defined herein, said method comprising performing the method as defined herein to obtain the BC suspension.

### DESCRIPTION OF DRAWINGS

FIG. 1. Physical appearance of paper which was coated with colored/uncolored bacterial suspension C (a), D (b), E (c), F (d), G (e) and H (f).

FIG. 2. Physical appearance of BC suspension D (a), MORFI of BC suspension D (b) and SEM picture of BC suspension E (c).

FIG. 3. Emulsification appearance of BC suspension D (a), plant microfibrillated cellulose (plant MFC) (b), carboxymethyl cellulose (CMC) (c) or xanthan gum (XG) (d) of liquid parrafin in 10 different concentrations of the polymers: The concentrations were from 0.1 to 1% in increments of 0.1%.

FIG. 4. Particle stabilization appearance of BC suspension D (a), plant MFC1 (b) or plant MFC2 (c) of activated charcoal in different concentration of polymer concentration.

FIG. 5. Fourier-transform infrared spectroscopy (FTIR) of non-modified BC (cellulose), modified BC with SiO₂ (cellulose-sylilate/cellulose-silanol on figure)), and Si—O—Si. BC is BC suspension D.

FIG. 6. Thermogravimetric analysis (TGA) analysis of unmodified BC and BC modified with modified SiO. H15 and H18 represents sylilate tail length. BC is BC suspension D.

FIG. 7. Schematic drawing of one-phase BC-SiO₂ coating for decreasing oxygen, water and grease absorption for paper/paperboard. SiO₂ refers to un- and/or modified SiO₂.

FIG. 8. Schematic drawing (top), and water contact angle pictures (bottom) of non-(A) and coated (B) paper sample with one-phase BC-SiO₂ coating for paper/paperboard. SiO₂ refers to un- and/or modified SiO₂.

### DETAILED DESCRIPTION

### Definitions

Accessible chemical groups—The accessible chemical groups of a material such as cellulose, e.g. the accessible hydroxyl groups of cellulose, are those accessible, i.e. available or susceptible, for modification, such as for example for functionalization by substitution or modification with a functional group. Methods for measuring the amount of accessible groups are known in the art, see for example Vaisanen et al. (2018) for methods for measuring the amount of accessible hydroxyl groups.

Comminution—Comminution is the reduction of solid materials from one average particle size to a smaller particle size, by crushing, grinding, cutting, vibrating or other processes. Comminution may for example be used for preparing a BC suspension from a BC material, whereby the BC material is comminuted into smaller pieces, i.e. into fiber clusters (also called particles, bundles or agglomerates). The type and method of comminution may impact the size and structure of said fibers clusters, which in turn may impact the properties of the suspension.

Crystallinity index (CI)—The crystallinity index, or CI, is used to describe the relative amount of crystalline material in cellulose. The CI is quantitative, and is defined as the volume fraction of crystallinity of one phase in a given sample. Methods for measuring the CI are well known in the art. For example, it can be measured using X-ray powder diffraction (XRD), solid state ¹¹C NMR, infrared (IR) spectroscopy and Raman spectroscopy. Methods using Fourier transform-IR spectroscopy (FTIR or FT-IR) determine the CI by measuring the relative peak heights or areas from raw spectrographic data. Methods for determining CI using Fourier transform-IR spectroscopy are known in the art (O'Connor et al., 1958). Proteins within a sample can make it difficult to measure the crystallinity and thus determine the CI for example when using FTIR. To circumvent this, the sample can be treated with alcalase to remove proteins that interfered with the crystallinity measurements e.g. when using FTIR for determining CI.

Degree of polymerization—the degree of polymerization (DP) is the number of monomeric units in a macromolecule or polymer or oligomer molecule. The degree of polymerization can be calculated from the viscosity, wherein the viscosity is measured according to the standardized method SCAN-C 15:62 using the equation below:

$$D {P}^{0.905} = 0.75 * n$$

wherein n is the viscosity measured according to SCAN-C 15:62, and DP is the degree of polymerization.

Fiber clusters—fiber clusters, i.e. particles, bundles or agglomerates, are the solid particles of BC which are present in a BC suspension. The method and degree of comminution of the BC material directly affects the size of the fiber clusters in the BC suspension. The size of the fiber clusters can be determined by methods known in the art, such as for example by MORFI analysis using a MORFI analyser, such as a MORFI LB01 system. This software performs a discrimination between fibers, shives, and fine elements through size criteria (length and width). Another method to measure fiber cluster size is laser diffraction (LD). LD is a well-known method in the art to analyse the dimensions such as size of particles. LD is based on the diffraction of a laser light/beam when it passes through a particle suspension. The smaller the particle size, the larger the diffraction angle of the laser beam will be. The particle size is calculated using a light scattering model, which can be either Fraunhofer or MIE. The MIE model is more precise for smaller particles (<25 μm), but requires the knowledge of the refractive and absorption index (also known as the real and the imaginary part of the refractive index) of both the sample and the solvent. The precision of the particle size distribution depends on how accurately the optical parameters are known.

Sterilization—Sterilisation refers herein to any process that removes, kills, inactivates, or deactivates life, i.e. living organisms such as a microorganism. The sterilisation process can be either a chemical or a physical process or treatment of a sample, suspension, culture medium and/or pellicle. Sterilization can be achieved through various means, including heat, chemical treatment, irradiation, high pressure, and/or filtration. Various methods for sterilisation of samples, suspensions, culture media and/or pellicles are well-known in the art.

Functionalization—Functionalization is the process of adding new functions, features, capabilities or properties to a material by changing the surface chemistry of the material. Functionalization of a material may for example make said material more suitable for certain applications. A material can for example be functionalized by modifying chemical groups at the surface of the material, i.e. the accessible groups, such as by introducing a functional group or substituting the chemical groups with a functional group. Functional groups include for example acetyl groups, alkyl groups, nitro groups, halides, aryls, and alkenes. Provided that the functional group is more hydrophobic compared to the chemical groups originally present at the surface of the material, such modification may increase the hydrophobicity of said material.

Grammage—grammage is the area density of a paper product, i.e. the mass per unit of area. It is expressed in terms of grams per square meter (g/m²).

Hydrophobicity—hydrophobicity is the physical property of a molecule that is seemingly repelled from a mass of water, or is not attracted to a mass of water. Hydrophobic molecules tend to be nonpolar, and thus, since water molecules are polar, hydrophobic molecules do not dissolve well among them. Water on hydrophobic surfaces will exhibit a high water contact angle.

Oxygen transmission rate—the oxygen transmission rate (OTR) is a measurement of the amount of oxygen gas that passes through a substance over a given period. OTR is expressed as a volume of oxygen that penetrates a given area in a one-day period. Methods for testing the OTR of a substance are well known in the art.

Water vapor transmission rate—the water vapor transmission rate (WVTR), also sometimes referred to as the water transmission rate, is a measurement of the amount of water vapor that passes through a substance or material over a given period. WVTR is expressed as a weight of water vapor that penetrates a given area in a one-day period, for example g/m²/day, g/m²/24 hr, or g/100 in²/24 hr. Methods for testing and analysing the WVTR of a substance or material are well-known in the art.

Porosity—the porosity, i.e. the air permeability, of a substrate, such as paper, indicates the amount of air that, as a result of a difference in air pressure on both sides, passes through a defined area of the substrate within 5 seconds. It is expressed in μm/Pa·s and/or in ml/min. The porosity can be measured using methods known in the art, using instruments such as a Bendtsen air permeability tester.

Smoothness—smoothness is a measure of the evenness or lack of contour of a material's surface such as of a paper's surface. Material or paper surface smoothness is often described in terms of material or paper surface roughness where a lower result indicates a smoother material or paper surface. Thus, the term smoothness is interchangeable with the term roughness regarding description of the evenness or lack of contour of a material's surface such as a paper's surface. Smoothness can be determined using the standard ISO 8792-2:2013: Paper and board. Determination of roughness/smoothness (air leak methods)—Part 2: Bendtsen method. The Bendtsen method measures the air flow rate, and the smoothness is expressed in millilitre of air per minute (ml/min). Smoothness evaluates the irregularities that disturb the ideal surface of the plane. This property is related to numerous parameters, such as paper composition.

Suspension—A suspension is a heterogeneous mixture of a fluid which contains solid particles sufficiently large for sedimentation. In otherwords, it is a heterogeneous mixture in which the solute particles do not dissolve, but get suspended throughout the bulk of the solvent, left floating around freely in the medium.

Viscosity—Viscosity (herein dynamic viscosity), is a measure of the viscosity of a fluid, i.e. a measure of its resistance to deformation at a certain rate. The higher the viscosity, the thicker the fluid; the lower the viscosity, the thinner the fluid. Viscosity can be measured using methods known in the art, such as for example with a rheometer. The viscosity is measured against the shear rate, and has a unit of Pa·s (·10⁻³ kg m⁻¹ s⁻¹).

Water absorption—the water absorption is the capacity of a plastic or a material to absorb moisture from its environment. The water absorption can be measured using the COBB test, where the amount of water (g/m²) absorbed into the surface by a sized paper, paperboard, or corrugated fibreboard paper during a set period of time is determined.

Water contact angle—the contact angle is the angle, conventionally measured though the liquid, where a liquid-vapor interface meets a solid surface. It quantifies the wettability of a solid surface by a liquid via the Young equation (see equation below). If the liquid is water, it is denoted the water contact angle. Generally, if the water contact angle is smaller than 90°, the solid surface is considered hydrophilic, and if the water contact angle is larger than 90°, the solid surface is considered hydrophobic.

$${SV}^{} = {S L}^{} + {LV}^{} cos θ$$

wherein γ^{SV} is the solid surface free energy; γ^{SL} is the solid/liquid interfacial free energy; γ^{LV} is the liquid surface free energy; and cos θ is the contact angle. The water contact angle or contact angle is sometimes also referred to as wetting angle.

Oil absorption—the oil absorption of a material is the capacity of said material such as plastic, paper, paperboard/cardboard or corrugated fibreboard paper to absorb oil or grease. The oil absorption can be measured using the COBB UNGER test, where the amount of oil (g/m²) absorbed into the surface of a material such as a sized paper, paperboard/cardboard, or corrugated fibreboard paper during a fixed period of time is determined.

Grease resistance—the grease resistance of a material is the degree of repellence and/or antiwicking characteristics to grease, oil or waxes of said material. The grease resistance of a material such as a paper, paperboard or board can be tested or measured by the so-called KIT test. The KIT test is often applied to test the grease resistance of materials such as paper, paperboard or board. The KIT number or rating of a material is the (rank) number representing the highest value of a series of test solutions that can be placed on the surface of said material without causing a wetting interaction such as darkening of the material surface by grease, oil or wax.

Cellulytic agent—a cellulytic agent is herein defined as an agent, compound or substance that can catalyse modification of cellulosic material such as cellulose (polymers) or matrix polysaccharides. Said modification can be decomposition, cleavage or hydrolyzation of the cellulosic material such as cellulose. The cellulytic agent may be a protein such as an enzyme or expansin. Said enzyme may be a cellulytic enzyme, such as a cellulase (EC 3.2.1.4), an endoglucanase (EC 3.2.1.6), a cellobiohydrolase (EC 3.2.1.91), an exoglucanase, or a beta-glucosidase. When the cellulytic agent is a protein, such as an enzyme, said enzyme may be any enzyme characterized by and/or associated with EC number 3.2.1, provided that said enzyme can catalyse modification of cellulosic material. Methods to analyse the activity of such enzymes catalysing modification of cellulosic material are well-known in the art, see for example Fapyane and Ferapontova (2017).

Silicon oxide—the term silicon oxide may herein be used to refer to either silicon monoxide and/or silicon dioxide.

The present invention provides a BC suspension and methods of producing the same. The inventors have found that BC suspensions with a certain solid content possess properties which are superior for coating applications. Furthermore, the inventors have discovered that functionalization of the produced BC material by substitution of OH groups on the surface of said material will render BC suspensions with improved coating properties.

Thus, presented herein are BC suspensions to be used for coating and methods of producing the same, wherein said BC suspensions offer improved mechanical and barrier properties for the coated materials.

Further, presented herein are BC suspensions to be used as particle stabilizers (particle stabilizators) and/or emulsion stabilizers (emulsifiers) and/or thickeners of suspensions and/or solutions.

### Bacterial Cellulose Suspension

Provided herein is a BC suspension comprising between 1.6 and 2.3% (w/v) of BC solids, such as 1.6% (w/v) of BC solids, such as 1.7% (w/v) of BC solids, such as 1.8% (w/v) of BC solids, such as 1.9% (w/v) of BC solids, such as 2.0% (w/v) of BC solids, such as 2.1% (w/v) of BC solids, such as 2.2% (w/v) of BC solids, such as 2.3% (w/v) of BC solids. Preferably, said BC suspension comprising BC fiber clusters having a size between 10 nm and 1500 μm, and/or said BC suspension having a viscosity between 1 and 500 Pa·s at a shear stress of 1/s.

In some embodiments, the BC suspension comprises between 1.6 and 2.3% (w/v) of BC solids, such as between 1.7 and 2.3% (w/v) of BC solids, such as between 1.8 and 2.3% (w/v) of BC solids, such as between 1.9 and 2.3% (w/v) of BC solids, such as between 2 and 2.3% (w/v) of BC solids, such as between 2.1 and 2.3% (w/v) of BC solids, such as between 2.2 and 2.3% (w/v) of BC solids. Further, in some embodiments the BC comprises between 1.6 and 2.2% (w/v) of BC solids, such as between 1.6 and 2.1% (w/v) of BC solids, such as between 1.6 and 2.0% (w/v) of BC solids, such as between 1.6 and 1.9% (w/v) of BC solids, such as between 1.6 and 1.8% (w/v) of BC solids, such as between 1.6 and 1.7% (w/v) of BC solids.

In some embodiments, the BC suspension comprises between 1.7 and 2.2% (w/v) of BC solids, such as between 1.7 and 2.1% (w/v) of BC solids, such as between 1.8 and 2.2% (w/v) of BC solids, such as between 1.7 and 2.0% (w/v) of BC solids, such as between 1.8 and 2.1% (w/v) of BC solids, such as between 1.8 and 2.0% (w/v) of BC solids, such as between 1.9 and 2.2% (w/v) of BC solids, such as between 1.9 and 2.1% (w/v) of BC solids, such as between 1.9 and 2.0% (w/v) of BC solids.

In some embodiments, the BC suspension comprises between 1.6 and 1.9% (w/v) of BC solids, such as between 1.6 and 1.8% (w/v) of BC solids, such as between 1.6 and 1.7% (w/v) of BC solids, such as 1.6% (w/v) of BC solids, such as 1.7% (w/v) of BC solids, such as 1.8% (w/v) of BC solids, such as 1.9% (w/v) of BC solids.

In some embodiments, the BC suspension comprises between 2.1 to 2.3% (w/v) of BC solids, such as between 2.1 and 2.2% (w/v) of BC solids, such as 2.1% (w/v) of BC solids, such as 2.2% (w/v) of BC solids, such as 2.3% (w/v) of BC solids.

In some embodiments, the BC suspension comprises between 1.6 and 1.9% (w/v) of BC solids, or between 2.1 and 2.3% (w/v) of BC solids, such as 1.6, 1.7, 1.8, 1.9, 2.1, 2.2 or 2.3% (w/v).

Also, provided herein is a BC suspension comprising 0.5 to 4% (w/v) of BC solids, such as 0.5% (w/v), such as 0.6% (w/v), such as 0.7% (w/v), such as 0.8% (w/v), such as 0.9% (w/v), such as 1.0% (w/v), such as 1.1% (w/v), such as 1.2% (w/v), such as 1.3% (w/v), such as 1.4% (w/v), such as 1.5% (w/v), such as 1.6% (w/v), such as 1.7% (w/v), such as 1.8% (w/v), such as 1.9% (w/v), such as 2.0% (w/v), such as 2.1% (w/v), such as 2.2% (w/v), such as 2.3% (w/v), such as 2.4% (w/v), such as 2.5% (w/v), such as 2.6% (w/v), such as 2.7% (w/v), such as 2.8% (w/v), such as 2.9% (w/v), such as 3.0% (w/v), such as 3.1% (w/v), such as 3.2% (w/v), such as 3.3% (w/v), such as 3.4% (w/v), such as 3.5% (w/v), such as 3.6% (w/v), such as 3.7% (w/v), such as 3.8% (w/v), such as 3.9% (w/v), such as 4.0% (w/v). In some embodiments, the BC suspension comprises between 0.5 and 4% (w/v) of BC solids, such as between 0.5 and 1% (w/v) of BC solids, such as between 1 and 2% (w/v) of BC solids, such as between 2 and 3% (w/v) of BC solids, such as between 3 and 4% (w/v) of BC solids.

The BC suspension comprises fiber clusters dispersed in a solvent, wherein the fiber clusters comprise solid BC particles. These fiber clusters may also be referred to as clusters, bundles, particles, and/or agglomerates of solid BC. The size of the BC fiber clusters of the BC suspension may vary within the BC suspension. It follows that the size of the BC fiber clusters may be the same and/or different from each other within the same and/or among different BC suspensions. In some embodiments, the size of the BC fiber clusters varies between 10 nm to 1500 μm, such as 10 nm to 1000 μm, such as 10 nm to 800 μm, such as 50 nm to 500 μm, such as 50 nm to 400 μm, such as 50 nm to 100 μm, such as 50 nm to 10 μm, such as 50 to 1000 nm, such as 50 to 100 nm, such as 10 to 100 μm, such as 100 nm to 100 μm, for example 10 nm, 75 nm, 500 nm, 900 nm, 2500 nm, 5000 nm, 15 μm, 50 μm, 250 μm, 600 μm, 900 μm, 1200 μm or 1500 μm. In some embodiments, the sizes of the BC fiber clusters varies between 0.5 and 1500 μm, such as between 0.5 and 1300 μm, such as between 1 and 200 μm, such as between 5 and 1400 μm, such as between 5 and 1350 μm, such as between 5 and 1300 μm, such as between 50 and 1400 μm, such as between 50 and 1350 μm, such as between 75 and 1300 μm, such as between 75 and 1300 μm, such as between 70 and 1250 μm, such as between 5 and 1240 μm.

In some embodiments, the median size of the BC fiber clusters varies between 25 and 150 μm, such as between 25 and 125 μm, such as between 25 and 100 μm, such as between 50 and 125 μm, such as between 50 and 100 μm, such as between 50 and 75 μm, such as between 75 and 100 μm, such as between 75 and 90 μm, such as between 80 and 100 μm, such as between 80 and 90 μm.

In some embodiments, the viscosity of the BC suspension is 2 to 500 Pa·s at a shear stress of 1/s, such as 2 Pa·s, such as 5 Pa·s, such as 10 Pa·s, such as 15 Pa·s, such as 20 Pa·s, such as 25 Pa·s, such as 30 Pa·s, such as 35 Pa·s, such as 40 Pa·s, such as 45 Pa·s, such as 50 Pa·s, such as 60 Pa·s, such as 70 Pa·s, such as 80 Pa·s, such as 90 Pa·s, such as 100 Pa·s, such as 150 Pa·s, such as 200 Pa·s, such as 250 Pa·s, such as 300 Pa·s, such as 350 Pa·s, such as 400 Pa·s, such as 450 Pa·s, such as 500 Pa·s. In some embodiments, the viscosity of the BC suspension is between 2 and 500 Pa·s, such as between 2 and 99 Pa·s, such as between 100 and 199 Pa·s, such as between 200 and 299 Pa·s, such as between 300 and 399 Pa·s, such as between 400 and 500 Pa·s at a shear stress of 1/s.

The BC suspension comprises BC solids, i.e. the BC fiber clusters, suspended in a solvent. In some embodiments, the solvent is a protic solvent, such as water, acetic acid, and/or ethanol. In a preferred embodiment, the solvent is water.

BC can be functionalized to introduce new properties to the material. Functionalization may for example change the surface chemistry of the BC, which may make it more suitable for certain applications. The BC material can for example be functionalized by modifying certain chemical groups at the surface of the material, i.e. the accessible groups, such as by introducing a functional group or substituting said chemical groups with a functional group. Functional groups include for example acetyl groups, alkyl groups, nitro groups, halides, aryls, and alkenes. Provided that the functional group is more hydrophobic compared to the chemical groups, e.g. hydroxyl groups, originally present at the surface of the BC, such modification may increase the hydrophobicity of the BC.

In some embodiments, the BC material comprised in the BC suspension has been contacted with a functionalization agent, whereby at least some of the accessible hydroxyl groups of the BC material have been functionalized. In some embodiments, the BC material is functionalized prior to preparing the BC suspension from the BC material. The functionalization agent may in some embodiments be selected from nitric acid and acetic anhydride and combinations thereof. In some embodiments contacting the BC material with the functionalization agent results in substitution of at least some of the accessible hydroxyl groups of the BC material with nitro groups. In other embodiments, contacting the BC material with the functionalization agent results in acetylation of at least some of the accessible hydroxyl groups of the BC material. In other embodiments, contacting the BC material with the functionalization agent results in modification of the BC material, wherein the modification is selected from substitution of at least some of the accessible hydroxyl groups of the BC material with nitro groups and acetylation of at least part of the accessible hydroxyl groups of the BC material. In some embodiments, 1 to 80% of the accessible groups are modified, such as 1%, such as 5%, such as 10%, such as 20%, such as 30%, such as 40%, such as 50%, such as 60%; such as 70%, such as 80%, such as 5 to 60%, such as 10 to 40%, such as 15 to 30%, such as 5 to 15%. In some embodiments, at least 1% of the accessible groups are modified, such as at least 5%, at least 10%, at least 15%, at least 20%, at least 25%, at least 30%, at least 35%, at least 40%, at least 45%, at least 50%, or more.

In some embodiments, the functionalization agent is nitric acid, and contacting the BC with nitric acid results in that at least part of the accessible hydroxyl groups of the BC are substituted with nitro groups. In some embodiments, 1 to 80% of the accessible hydroxyl groups are substituted with nitro groups, such as 1%, such as 5%, such as 10%, such as 20%, such as 30%, such as 40%, such as 50%, such as 60%; such as 70%, such as 80%, such as 5 to 60%, such as 10 to 40%, such as 15 to 30%, such as 5 to 15%. In some embodiments, at least 1% of the accessible hydroxyl groups are substituted with nitro groups, such as at least 5%, at least 10%, at least 15%, at least 20%, at least 25%, at least 30%, at least 35%, at least 40%, at least 45%, at least 50%, or more.

In some embodiments, the functionalization agent is acetic anhydride, and contacting the BC with acetic anhydride results in that at least part of the accessible hydroxyl groups of the BC are acetylated. In some embodiments, 1 to 80% of the accessible hydroxyl groups are acetylated, such as 1%, such as 5%, such as 10%, such as 20%, such as 30%, such as 40%, such as 50%, such as 60%; such as 70%, such as 80%, such as 5 to 60%, such as 10 to 40%, such as 15 to 30%, such as 5 to 15%. In some embodiments, at least 1% of the accessible hydroxyl groups are acetylated, such as at least 5%, at least 10%, at least 15%, at least 20%, at least 25%, at least 30%, at least 35%, at least 40%, at least 45%, at least 50%, or more.

The BC suspension may further comprise one or more further compounds, such as a water-soluble or a water-insoluble component. The one or more further compounds may improve and/or change certain properties of the suspension, such as for example the stability, viscosity and/or the dispersibility of said suspension. The one or more further compounds may also be referred to as an additional component and/or additional components. Examples of such compounds include glycerin, ethylene glycol, dimethyl sulfoxide, dimethylformamide, lactic acid, gluconic acid and delta gluconolactone, a low molecular mass saccharide (e.g. glucose, fructose, galactose, xylose, mannose, arabinose, sucrose, lactose, cellobiose, palatinose, maltose, gentiobiose, trehalose, rhamnose, isomalto-oligosaccharide, soybean oligosaccharide, fructo-oligosaccharide, galactooligosaccharide, lactosucrose, coupling sugar, liquid sugar, cyclodextrin, sugar alcohols, sorbitol, erythritol, lactitol, maltitol, xylitol, mannitol, dulcit), salts (sodium sulfate, ammonium sulfate, sodium chloride, calcium chloride, sodium bicarbonate, sodium carbonate, Rossel salt), aminoacids, amino acid salts, acidic acids, organic acids, nucleic acids, nucleic acid salts, high molecular mass polysaccharides (e.g. xanthan gum, xyloglucan, dextrin, dextran, carrageenan, locust bean gum, tamarind gum, alginic acid, alginate, pullulan, starch, starch, CMC, methylcellulose, hydroxypropylcellulose, ethylcellulose, corn starch, gum arabic, guargum, gellan gum, polydextrose, pectin), chitin, chitosan, casein, alpimine, soybean protein lysate, peptone, polyvinyl alcohol, polyacrylamide, polyacrylic acid sodium polyvinylpyrrolidone, a polyamide or polyamideimine or polyamine resin, polyethylene oxide, hydrophilic crosslinked polymer, polyacrylic acid copolymer, unmodified or modified silicon oxide, titanium oxide, and colloidal silica. In one embodiment, the BC suspension comprises polyvinyl alcohol (PVOH). In some embodiments the silicon oxide is silicon dioxide (SiO₂). In some embodiments the unmodified or modified silicon oxide is unmodified or modified silicon dioxide (SiO₂). In some embodiments, the BC suspension comprises unmodified and/or modified silicon dioxide (SiO₂). In some embodiments, the modified silicon dioxide (SiO₂) is silicon dioxide (SiO₂) modified with dimethylsylilates particles having a sylilate tail length of H8 to H22, such as a sylilate tail length of H10 to H20, such as a sylilate tail length of H15 to H20, such as sylilate tail length of H15 to H18, such as a sylilate tail length of H15, such as a sylilate tail length of H16, such as a sylilate tail length of H17, such as a sylilate tail length of H18. In some embodiments, the modified silicon dioxide (SiO₂) is selected from SiO₂ modified with dimethylsylilates H15 particles with BET surface of 130-170 m²/g and SiO₂ modified with dimethylsylilates H18 particles with BET surface of 170-230 m²/g.

In some embodiments, the BC suspension further comprises from 0.1 to 5% polyvinyl alcohol (PVOH), such as 0.2-5%, such as 0.3-5%, such as 0.4-5%, such as 0.5-5%, such as 0.1-2.5%, such as 0.1-2%, such as 2.5-5%, such as 2-3%, such as 3-4%, such as 4-5%. In some embodiments, the BC suspension further comprises up to 5% PVOH, such as 0.1%, such as 0.5%, such as 0.75%, such as 1%, such as 1.5%, such as 2%, such as 3%, such as 4%, such as 5% PVOH. In some embodiments, the BC suspension comprises up to 1% PVOH. In some embodiments, the BC suspension comprises 1% PVOH.

The BC suspension presented herein may be used for coating a material, such as paper, paperboard, food stuff such as produce, cosmetics, and/or textiles, such as woven and/or non-woven textiles. In one embodiment, coating a paper or a paperboard with the BC suspension disclosed herein results in a coated material with a specific grammage, porosity, smoothness, water absorption, and/or OTR.

Thus, presented herein are methods of coating a material, such as the surface of a material, with the BC suspension, thereby obtaining a coated material.

Further presented herein are methods of preparing a multi-layer coating of a material, wherein the material is coated with a BC suspension after being coated with a further coating agent, or wherein the material is coated with another coating agent after being coated with a BC suspension. Such multi-layer coating can improve the properties of the coated material, such as the porosity, smoothness, water absorption and/or the oxygen transmission rate (OTR). The further coating agent can be any agent, such as for example polyvinyl alcohol (PVOH), nitrocellulose and/or silicon oxide.

Presented herein are also methods of preparing a one-phase coating of a material, wherein the material is coated with a BC suspension, said BC suspension may comprise one or more compounds such as an additional component. The one-phase coating of a material is different from multi-layer coating since it needs be applied on said material only once. Such one-phase coating can improve the properties of the coated material, such as the porosity, smoothness (or roughness), water absorption, water contact angle, oxygen transmission rate (OTR), water vapor transmission rate (WVTR), grease resistance, and/or oil absorption. The additional component may be any compound, such as for example polyvinyl alcohol (PVOH), modified and/or unmodified silicon oxide. The one-phase coating comprises BC suspended in a solvent. In some embodiments, the one-phase coating may be a water-based one-phase coating wherein water is used as solvent. In some embodiments, the solvent of the one-phase coating is a polar solvent.

In other words, the BC suspension presented herein can be used for coating any type of material, on its surface, either an inner surface or an outer surface.

In some embodiments, the material is selected from the group of materials consisting of paper, paperboard/cardboard, foodstuff, cosmetics, and textiles, such as woven and/or non-woven textiles. In some embodiments, the material is specialty paper, corrugated fibreboard paper, cellulose- and/or fiber-based materials. In some embodiments, the material is textile, such as woven and/or non-woven textiles.

Coating of a material may improve the properties of said material. For example, coating can make a material more resistance to water and damage. When the material is a perishable material, e.g. food stuff or a cosmetic product, the coating may alternatively or additionally prolong the shelf-life of the material. The coating can be applied in one or several layers, and the thickness of the coating can vary.

There are several methods that can be used to coat a material. These include but are not limited to spray coating, reverse roll coating, immersion (dip) coating, lamination coating, knife over roll coating, gravure coating, and/or curtain coating. Certain instruments, such as for example an automatic film applicator, can also be used for coating.

The thickness of the coating, i.e. the coating thickness can vary. The coating thickness is the thickness of the coating after the coat and the coated material have dried, i.e. the dry film thickness or dry coating thickness. The coating thickness can be measured using methods known in the art, such as for example with a coating thickness gauge. Two readings are made with the coating thickness gauge; one before and one after the coating has been applied. The difference between the two readings, i.e. the height variation, equals the coating thickness.

In some embodiments of the present invention, the coating thickness of the BC suspension is between 30 and 110 μm, such as 30 μm, such as 40 μm, such as 50 μm, such as 60 μm, such as 70 μm, such as 80 μm, such as 90 μm, such as 100 μm, such as 110 μm, such as between 30 and 80 μm, such as between 50 and 90 μm, such as between 60 and 100 μm, such as between 40 and 70 μm. In some embodiments of the present invention, the coating thickness of the BC suspension is between 1 and 40 μm, such as between 1 and 10 μm, such as 1 μm, such as 2 μm, such as 3 μm, such as 4 μm, such as between 5 and 10 μm, such as 5 μm, such as 6 μm, such as 7 μm, such as 8 μm, such as 9 μm, such as 10 μm, such as between 10 and 20 μm, such as 12 μm, such as 14 μm, such as 16 μm, such as 18 μm, such as 20 μm, such as between 20 and 40 μm, such as between 20 and 30 μm, such as 22 μm, such as 24 μm, such as 26 μm, such as 28 μm, such as 29 μm, such as between 30 and 40 μm. In some embodiments the coating thickness of the BC suspension is at the most 10 μm, such as less than 10 μm, such as between 1 and 10 μm. In some embodiments the coating thickness of the BC suspension is at the most 10 μm.

It is also possible to measure the wet coating thickness. The wet coating thickness is the height of the wet coating material, i.e. the wet BC suspension, which is deposited to the material to be coated. In other words, the wet coating thickness is the thickness of the coating before said coating has dried. It can be measured with methods known in the art, such as with a wet film thickness gauge.

In some embodiments, the wet coating thickness of the BC suspension is between 20 and 80 μm, such as 20 μm, such as 30 μm, such as 40 μm, such as 50 μm, such as 60 μm, such as 70 μm, such as 80 μm, such as between 20 and 60 μm, such as between 30 and 50 μm, such as between 40 and 70 μm.

In some embodiments, the wet coating thickness of the BC suspension is between 10 and 150 μm, such as between 25 and 125 μm, such as between 30 and 100 μm, such as 10 μm, such as 20 μm, such as 30 μm, such as 40 μm, such as 50 μm, such as 60 μm, such as 70 μm, such as 80 μm, such as 100 μm, such as 110 μm, such as 120 μm, such as 130 μm, such as 140 μm, such as 150 μm, such as between 10 and 50 μm, such as between 30 and 75 μm, such as between 50 and 110 μm, such as between 75 and 150 μm, such as between 90 and 100 μm. In preferred embodiments, the wet coating thickness of the BC suspension is 100 μm.

Coating of paper and cardboard/paperboard can impact and/or improve the weight, smoothness, porosity, water resistance, OTR, WVTR, oil absorption, grease resistance, and/or other properties of the paper.

In some embodiments, coating a paper or a cardboard with the BC suspension generates a coated paper or cardboard with a grammage between 45 and 70 g/m², such as 45 g/m², such as 46 g/m², such as 47 g/m², such as 48 g/m², such as 49 g/m², such as 50 g/m², such as 51 g/m², such as 52 g/m², such as 53 g/m², such as 54 g/m², such as 55 g/m², such as 56 g/m², such as 57 g/m², such as 58 g/m², such as 59 g/m², such as 60 g/m², such as 61 g/m², such as 62 g/m², such as 63 g/m², such as 64 g/m², such as 65 g/m², such as 66 g/m², such as 67 g/m², such as 68 g/m², such as 69 g/m², such as 70 g/m². In some embodiments, the grammage is between 45 and 50 g/m², or between 50 and 55 g/m², or between 55 and 60 g/m², or between 60 and 65 g/m², or between 65 and 70 g/m².

In some embodiments, coating a paper or a cardboard with the BC suspension generates a coated paper or cardboard with a grammage between 25 and 500 g/m², such as between 40 and 450 g/m², such as between 40 and 400 g/m², such as between 50 and 400 g/m², such as between 50 and 375 g/m², such as between 50 and 360 g/m².

In some embodiments, coating a paper or a cardboard with the BC suspension generates a coated paper or cardboard with a smoothness between 200 and 1000 mL/min, such as 200 mL/min, such as 250 mL/min, such as 300 mL/min, such as 350 mL/min, such as 400 mL/min, such as 450 mL/min, such as 500 mL/min, such as 550 mL/min, such as 600 mL/min, such as 700 mL/min, such as 800 mL/min, such as 900 mL/min, such as 1000 mL/min, for example between 200 and 500 mL/min, between 300 and 600 mL/min, between 400 and 700 mL/min, between 500 and 1000 mL/min, between 600 and 900 mL/min, or between 400 and 800 mL/min. The smoothness of a coated paper or cardboard might also sometimes be illustrated by referring to the roughness of a coated paper or cardboard.

In some embodiments, coating a paper or a cardboard with the BC suspension generates a coated paper or cardboard with a porosity between 50 and 250 mL/min, such as 50 mL/min, such as 60 mL/min, such as 70 mL/min, such as 80 mL/min, such as 90 mL/min, such as 100 mL/min, such as 110 mL/min, such as 120 mL/min, such as 130 mL/min, such as 140 mL/min, such as 150 mL/min, such as 175 mL/min, such as 200 mL/min, such as 225 mL/min, such as 250 mL/min, such as between 65 and 105 mL/min, such as between 95 and 150 mL/min, such as between 75 and 175 mL/min, such as between 100 and 200 mL/min, such as between 50 and 145 mL/min.

In some embodiments, coating a paper or a cardboard with the BC suspension generates a coated paper or cardboard with a porosity between 1 and 250 mL/min, such as between 1 and 200 mL/min, such as between 1 and 175 mL/min, such as between 1 and 150 mL/min, such as between 2.5 and 250 mL/min, 2.5 and 200 mL/min, such as between 2.5 and 175 mL/min, such as between 2.5 and 150 mL/min, such as between 5 and 250 mL/min, 5 and 200 mL/min, such as between 5 and 175 mL/min, such as between 5 and 150 mL/min.

In some embodiments, coating a paper or a cardboard with the BC suspension generates a coated paper or cardboard with a water absorption of 15 to 25 g/m², such as 15 16 g/m², such as 16 g/m², such as 17 g/m², such as 18 g/m², such as 19 g/m², such as 20 g/m², such as 21 g/m², such as 22 g/m², such as 23 g/m², such as 24 g/m², such as 16 g/m². In some embodiments, the water absorption is between 16 and 24 g/m², such as between 17 and 23 g/m², such as between 18 and 22 g/m², such as between 19 and 21 g/m².

In some embodiments, coating a paper or a cardboard with the BC suspension generates a coated paper or cardboard with an oxygen transmission rate (OTR) between 1 and 60 ml/m²·day·atm, such as between 2 and 7 ml/m²·day·atm, such as between 15 and 50 ml/m²·day·atm, such as between 10 and 25 ml/m²·day·atm, such as 3 and 5 ml/m²·day·atm.

In some embodiments, coating a paper or a cardboard with the BC suspension generates a coated paper or cardboard with a water vapor transmission rate (WVTR) less than 175 g/m²/day, such as less than 150 g/m²/day, such as less than 125 g/m²/day, such as 100 g/m²/day, such as less than 75 g/m²/day, such as less than 60 g/m²/day, such as less than 50 g/m²/day, such as less than 45 g/m²/day, such as less than 40 g/m²/day, such as less than 30 g/m²/day, such as less than 25 g/m²/day, such as less than 20 g/m²/day, such as less than 15 g/m²/day, such as less than 10 g/m²/day, or less. In some embodiments, coating a paper or a cardboard with the BC suspension generates a coated paper or cardboard with a WVTR that is two to six times lower than the WVTR of the paper or cardboard before the coating, such as two times, such as three times, such as four times, such as five times, such as six times lower compared to the WVTR of the paper or cardboard before the coating.

In some embodiments, coating a paper or a cardboard with the BC suspension generates a coated paper or cardboard with an oil absorption less than 45 g/m², such as less than 40 g/m², such as less than 30 g/m², such as less than 20 g/m², such as less than 15 g/m², such as less than 10 g/m², such as less than 9.5 g/m², such as less than 9 g/m², such as less than 8.5 g/m², such as less than 8 g/m², such as less than 8 g/m², such as less than 7.5 g/m², such as less than 7 g/m², or less. In some embodiments, coating a paper or a cardboard with the BC suspension generates a coated paper or cardboard with an oil absorption that is two to six times lower than the oil absorption of the paper or cardboard before the coating, such as two times, such as three times, such as four times, such as five times, such as six times lower compared to the oil absorption of the paper or cardboard before the coating.

In some embodiments, coating a paper or a cardboard with the BC suspension generates a coated paper or cardboard with a grease resistance of a KIT number of at least 6, such as at least 7, such as at least 8, such as at least 9, such as at least 10, such as at least 11, such as 12. In some embodiments, coating a paper or a cardboard with the BC suspension generates a coated paper or cardboard with a grease resistance that is two to six KIT points/numbers higher than the grease resistance of the paper or cardboard before the coating when said grease resistance is measured or tested using the KIT test, such as two KIT points/numbers, such as three KIT points/numbers, such as four KIT points/numbers, such as five KIT points/numbers, such as six KIT points/numbers higher compared to the grease resistance of the paper or cardboard before the coating.

The BC suspension presented herein may be applied and/or used as particle stabilizer (particle stabilizator) and/or emulsion stabilizer (emulsifier) and/or thickener (thickening agent) of suspensions, solutions and/or liquids. In some embodiments, the BC suspension disclosed herein is used as particle stabilizer (particle stabilizator) of particles in a suspension, solution and/or liquid, which results in a more stable suspension, solution and/or liquid mixed with said particles compared to mixing said particles with the solution, suspension, and/or liquid without said BC suspension. In some embodiments, the BC suspension disclosed herein is used as an emulsion stabilizer (emulsifier) to stabilize an emulsion such as by increasing the kinetic stability of said emulsion. Thus, in some embodiments, the BC suspension disclosed herein is used as an emulsion stabilizer, whereby suspension of one liquid in another liquid is encourage, improved and/or aided. In some embodiments, the BC suspension disclosed herein is used as thickener of a suspension, solution and/or liquid, which results in a more thick solution, suspension and/or liquid compared to the solution, suspension and/or liquid, wherein said BC suspension is not used. Thus, in some embodiments the BC suspension disclosed herein is used as thickener of a suspension, solution and/or liquid, whereby the viscosity of said suspension, solution and/or liquid is increased.

In some embodiments, when applied to coat a paper of a grammage of 60 g/m² at a dry coating thickness of 40 μm, the BC suspension results in a water vapor transmission rate (WVTR) range of 50-200 g/m²/day at 50% relative humidity, such as 75-175 g/m²/day at 50% relative humidity, such as 85-160 g/m²/day at 50% relative humidity, such as 90-155 g/m²/day at 50% relative humidity, such as 90.8-154.6 g/m²/day at 50% relative humidity.

In some embodiments, when applied to coat a paper of a grammage of 60 g/m² at a dry coating thickness of 40 μm, the BC suspension results in an oxygen transfer rate (OTR) range of 2.5-40 ml/m²/day/atm, such as 5-35 ml/m²/day/atm, such as 7.5-30 ml/m²/day/atm, such as 10-26 ml/m²/day/atm, such as 11.2-24.2 ml/m²/day/atm.

### Method for Producing a Bacterial Cellulose Suspension

Further provided herein is a method for producing a BC suspension, comprising the steps of:
a) Incubating at least one cellulose-producing bacteria in a culture medium, and optionally, at least one other microorganism which does not produce cellulose, thereby obtaining BC in said culture medium;
b) Recovering said BC;
c) Optionally, contacting said BC with a functionalization agent, thereby functionalizing said BC;
d) Converting the recovered and/or the functionalized BC into a BC suspension comprising 0.5 to 4.0% (w/v) BC solids.

Also provided herein is a method for producing a BC suspension, comprising the steps of:
a) Incubating at least one cellulose-producing bacteria in a culture medium, and optionally, at least one other microorganism which does not produce cellulose, thereby obtaining BC in said culture medium;
b) Recovering said BC;
c) Optionally, contacting said BC with a functionalization agent, thereby functionalizing said BC;
d) Converting the recovered BC of step b) and/or the functionalized BC of step c) into a BC suspension comprising 1.6 to 2.3% (w/v) BC solids. Preferably, said BC suspension comprising BC fiber clusters having a size between 10 nm and 1500 μm, and/or said BC suspension having a viscosity between 1 and 500 Pa·s at a shear stress of 1/s.

In some embodiments, the method further comprises a step of sterilisation, preferably said step is performed before step a to d. Preferably, said step is performed before step c.

In some embodiments, the method further comprises further incubating at least one microorganism producing or being capable of producing at least one cellulytic agent in said culture medium.

In some embodiments, the method further comprises a step of reacting and/or mixing the BC suspension and/or the BC, such as the recovered and/or functionalized BC, with one or more compounds such as an additional component. Said one or more compounds, such as an additional component may be glycerol, polyvinyl alcohol (PVOH), pectin, casein, starch, modified and/or unmodified silicon dioxide, and/or titanium oxide.

In some embodiments, the BC suspension comprises between 1.6 and 2.3% (w/v) of BC solids, such as between 1.7 and 2.3% (w/v) of BC solids, such as between 1.8 and 2.3% (w/v) of BC solids, such as between 1.9 and 2.3% (w/v) of BC solids, such as between 2 and 2.3% (w/v) of BC solids, such as between 2.1 and 2.3% (w/v) of BC solids, such as between 2.2 and 2.3% (w/v) of BC solids. Further, in some embodiments the BC comprises between 1.6 and 2.2% (w/v) of BC solids, such as between 1.6 and 2.1% (w/v) of BC solids, such as between 1.6 and 2.0% (w/v) of BC solids, such as between 1.6 and 1.9% (w/v) of BC solids, such as between 1.6 and 1.8% (w/v) of BC solids, such as between 1.6 and 1.7% (w/v) of BC solids.

In some embodiments, the BC suspension comprises between 1.7 and 2.2% (w/v) of BC solids, such as between 1.7 and 2.1% (w/v) of BC solids, such as between 1.8 and 2.2% (w/v) of BC solids, such as between 1.7 and 2.0% (w/v) of BC solids, such as between 1.8 and 2.1% (w/v) of BC solids, such as between 1.8 and 2.0% (w/v) of BC solids, such as between 1.9 and 2.2% (w/v) of BC solids, such as between 1.9 and 2.1% (w/v) of BC solids, such as between 1.9 and 2.0% (w/v) of BC solids.

In some embodiments, the BC suspension comprises between 1.6 and 1.9% (w/v) of BC solids, such as between 1.6 and 1.8% (w/v) of BC solids, such as between 1.6 and 1.7% (w/v) of BC solids, such as 1.6% (w/v) of BC solids, such as 1.7% (w/v) of BC solids, such as 1.8% (w/v) of BC solids, such as 1.9% (w/v) of BC solids.

In some embodiments, the BC suspension comprises between 2.1 to 2.3% (w/v) of BC solids, such as between 2.1 and 2.2% (w/v) of BC solids, such as 2.1% (w/v) of BC solids, such as 2.2% (w/v) of BC solids, such as 2.3% (w/v) of BC solids.

In some embodiments, the BC suspension comprises between 1.6 and 1.9% (w/v) of BC solids, or between 2.1 and 2.3% (w/v) of BC solids, such as 1.6, 1.7, 1.8, 1.9, 2.1, 2.2 or 2.3% (w/v).

Thus, in one embodiment, is provided a BC suspension obtained from the method presented herein. In some embodiments, the BC suspension is as defined in the section “Bacterial cellulose suspension”.

### Culture Medium

The culture medium may be any culture medium suitable for the purpose of producing BC. Preferably, the culture medium comprises a carbon source, such as glycerol, fructose, sucrose and/or glucose. The carbon source may also comprise ethanol and/or other sugar alcohols, alone or in combination with those previously mentioned. The inventors have found that the carbon source may affect the properties of the produced BC, such as the CI and/or the viscosity of the BC. For example, production of BC in a culture medium comprising sucrose and/or glucose as the main carbon source provides a BC material with a higher CI and/or viscosity compared to BC produced in a culture medium comprising glycerol and/or fructose as the main carbon source, wherein the production otherwise takes place under identical or similar conditions.

Further, the concentration of said carbon source in said culture medium may be any concentration suitable for the production process. In some embodiments, the culture medium comprises a carbon source at a concentration of 1 to 30%, such as 1%, such as 2%, such as 5%, such as 10%, such as 15%, such as 20%, such as 25%, such as 30%. In some embodiments, the culture medium comprises a carbon source at a concentration between 2 and 10%. In some embodiments, the culture medium comprises glucose at a concentration of 1 to 30%, such as 2%, such as 5% or more, such as 10% or more, such as 15% or more, such as 20% or more, such as 25% or more, such as 2 to 10%, such as 2 to 5%, such as 1 to 8%; such as 4 to 10%, such as 8 to 15%, such as 10 to 25%, such as 15 to 20%. In other embodiments, the culture medium comprises sucrose at a concentration of 1 to 30%, such as 2%, such as 5%, such as 10%, such as 15%, such as 20%, such as 25%, such as 2 to 10%, such as 2 to 5%, such as 1 to 8%; such as 4 to 10%, such as 8 to 15%, such as 10 to 25%, such as 15 to 20%.

The culture medium may be a defined medium, such as a chemically defined medium wherein which all or most of the chemical components are known. In some embodiments, the medium is Hestrin-Schramm (HS) medium.

The medium may also be prepared from molasses, plant waste, wood hydrolysate, straw hydrolysate, and/or from other sources of inexpensive biomass wastes suitable for the production of BC. Thus, in some embodiments, the culture medium comprises biomass waste, such as molasses and/or plant waste.

The culture medium may also comprise juice from vegetables and/or fruits, such as juice from apple, strawberry, blackberry, aronia, chokeberry, grape, plum, cranberry, pea, orange, banana, potato, carrot, sugar beet and/or combinations thereof. In some embodiments, the carbon source used in the culture medium is extracted from vegetables and/or fruits, such as from apple, strawberry, blackberry, aronia, chokeberry, grape, plum, cranberry, pea, orange, banana, potato, carrot, sugar beet and/or combinations thereof.

In addition, several nitrogen sources, organic or inorganic ones, can be comprised in the culture medium, such as ammonium sulphate, ammonium chloride, ammonium phosphate, urea, sodium nitrate, yeast extract, corn steep liquor, whey hydrolysate, peptone, casein hydrolysate and/or combinations thereof.

Additives may be added to the culture medium. Such additives include, but are not limited to, any additives which may be deemed beneficial for the production process. In some embodiments, the culture medium comprises one or more antioxidants and/or polyphenols extracted from fruits, vegetables, and/or tea, such as black tea. In some embodiments, the culture medium comprises theaflavins or derivatives thereof, wherein the derivative preferably comprises a tropolone moiety. In some embodiments, the culture medium comprises theaflavins selected from the group consisting of theaflavin, theaflavin-3-gallate, theaflavin-3′-gallate, and theaflavin-3-3′-digallate and combinations thereof.

In one embodiment, the culture medium comprises one or more polyphenols, such as theaflavins, at a concentration between 5 and 100 g/L, such as 5 g/L, such as 10 g/L, such as 20 g/L, such as 30 g/L, such as 40 g/L, such as 50 g/L, such as 60 g/L, such as 70 g/L, such as 80 g/L, such as 90 g/L, such as 100 g/L, for example between 5 and 90 g/L, such as between 10 and 80 g/L, for example between 15 and 75 g/L, such as between 20 and 70 g/L, for example between 25 and 65 g/L, such as between 30 and 60 g/L, for example between 35 and 55 g/L, such as between 40 and 50 g/L, for example about 45 g/L.

In order to avoid contamination, sterilization (such as through autoclavation), of the culture medium is normally carried out prior to microbial production of various compounds, i.e. prior to fermentation. However, sterilization of culture media is expensive and time-consuming, and may decrease the overall productivity of the microbial production process. Thus, it may be advantageous to provide a production method wherein the culture media does not need to be sterilized prior to fermentation, for example by conducting the fermentation at a low pH to minimize the risk of contamination. Hence, in one embodiment, the culture medium is not sterilized prior to incubating the at least one cellulose-producing bacteria, and optionally, the at least one other microorganism which does not produce cellulose, in said culture medium. In one embodiment, the culture medium has a pH value between 2.5 and 4.0, such as 2.5, such as 2.6, such as 2.7, such as 2.8, such as 2.9, such as 3.0, such as 3.1, such as 3.2, such as 3.3, such as 3.4, such as 3.5, such as 3.6, such as 3.7, such as 3.8, such as 3.9, such as 4.0. In some embodiments the pH is between 2.5 and 4.0, such as between 2.6 and 3.9, such as between 2.7 and 3.8, such as between 2.8 and 3.7, such as between 2.9 and 3.6, such as between 3.0 and 3.5, such as between 3.1 and 3.4, such as between 3.2 and 3.3.

### Microorganisms

The present invention concerns production of BC in a cellulose-producing bacteria. Bacteria which produce BC include for example those of the genus Acetobacter, Achromobacter, Aerobacter, Agrobacteria, Alcaligenes, Azotobacter, Gluconacetobacter, Gluconobacter, Komagataeibacter, Escherichia, Rhizobium, Pseudomonas, Salmonella and Sarcine. The most productive BC producing strains are of the genus Acetobacter, such as A. xylinum; Komagataeibacter, such as K. rhaeticus, and Gluconobacter, such as G. oxydans. Thus, in one embodiment, the at least one cellulose-producing bacteria is Komagataeibacter rhaeticus. In another embodiment, the at least one cellulose-producing bacteria is Gluconobacter oxydans. In yet another embodiment, the at least one cellulose-producing bacteria is at least two cellulose-producing bacteria, such as Komagataeibacter rhaeticus and Gluconobacter oxydans.

According to the present invention, at least one BC-producing bacteria may be used for production of BC, such as at least two or at least three BC-producing bacteria. Certain properties of the produced BC may depend on the bacteria used for production, such as the porosity and/or the degree of polymerization of the BC.

For the purpose of the present invention, a co-culture, i.e. a symbiotic culture, of microorganisms may be used for production of the BC. The use of co-cultures, such as cultures from Kombucha tea, may provide advantages over single-cell cultures, including higher BC yields and the possibility of running the fermentation at lower pH, minimizing the risk of contamination and enabling production in non-sterilized media. The co-culture may comprise microorganisms which produce BC, and microorganisms which do not produce BC, such as at least one cellulose-producing bacteria and optionally, at least one microorganism which does not produce cellulose. In some embodiments, the co-culture is isolated from a Kombucha tea biofilm. In other words, the at least one cellulose-producing bacteria and optionally, the at least one microorganism which does not produce cellulose, is isolated from a Kombucha tea biofilm.

Non-cellulose-producing microorganisms which may be isolated from a Kombucha tea biofilm may for example be of the genus Brettanomyces, Candida, Dekkera, Hanseniaspora, Kloeckera, Saccharomyces Mycotorula, Mycoderma, Pichia, Saccharomycodes, Schizosaccharomyces, Torulospora, and/or Zygosaccharomyces, such as Brettanomyces bruxellensis, Hanseniaspora uvarum, Kloeckera lindneri, and/or Zygosaccharomyces bailii.

In some embodiments, the at least one cellulose-producing bacteria is Komagataeibacter rhaeticus and/or Gluconobacter oxydans, and the at least one microorganism which does not produce cellulose is Brettanomyces bruxellensis, Hanseniaspora uvarum, Kloeckera lindneri, and/or Zygosaccharomyces bailii.

In one embodiment, the at least one cellulose-producing bacteria is Komagataeibacter rhaeticus and/or Gluconobacteroxydans, and the at least one microorganism which does not produce cellulose is at least two microorganisms which do not produce cellulose, such as at least two of Brettanomyces bruxellensis, Hanseniaspora uvarum, Kloeckera lindneri, and/or Zygosaccharomyces bailii, such as at least three or such as at least all four of Brettanomyces bruxellensis, Hanseniaspora uvarum, Kloeckera lindneri, and/or Zygosaccharomyces bailii.

The co-culture may further comprise at least one microorganism, for example a bacteria, which produces or are capable of producing at least one cellulytic agent. Thus, in some embodiments at least one microorganism, such as at least two, such as at least three, such as at least four, such as at least five, or more, which produces or is capable of producing at least one cellulytic agent may be applied.

Further, in some embodiments, the at least one microorganism produces at least one cellulytic agent, such as at least two, such as at least three, such as at least four, such as at least five, such as at least six, such as at least seven, such as at least eight, such as at least nine, such as at least 10, such as at least 11, such as at least 12, such as at least 13, such as at least 14, such as at least 15, such as at least 16, or more cellulytic agents.

In some embodiments the at least one cellulytic agent is a protein such as an enzyme or expansin. In some embodiments, said enzyme is a cellulytic enzyme. In some embodiments, said enzyme is a cellulase (EC 3.2.1.4), an endoglucanase (EC 3.2.1.6), a cellobiohydrolase (EC 3.2.1.91), an exoglucanase, or a beta-glucosidase. In some embodiments, said enzyme is an enzyme characterized by and/or associated with EC number 3.2.1.

The at least one cellulytic agent may be produced by an engineered and/or genetically modified microorganism or by a non-modified and/or non-engineered organism. Said microorganism might be a bacterium, fungus, yeast, archae, or another microorganism. In some embodiments, said microorganism is a bacterium that belongs to the Bacillus genus. In some embodiment, said bacterium is a Bacillus altitudinis bacterium.

In some embodiments, the at least one bacteria which produce BC and/or the at least one microorganism which does not produce BC is an engineered and/or genetically modified organism.

In some embodiments, the co-culture may comprise at least one bacteria which produce BC, and optionally at least one microorganism which does not produce BC, such as at least one cellulose-producing bacteria, and optionally at least one microorganism which does not produce cellulose and/or at least one microorganism which produces or is capable of producing at least one cellulytic agent. In some embodiments, the at least one microorganism which does not produce cellulose and the at least one other microorganism which produces or are capable of producing at least one cellulytic agent are the same. In some embodiments, the at least one microorganism which does not produce cellulose and the at least one other microorganism which produces or are capable of producing at least one cellulytic agent are different. In some embodiments, the at least one cellulose-producing bacteria and the at least one other microorganism which produces or are capable of producing at least one cellulytic agent are the same. In some embodiments, the co-culture may comprise at least one bacteria which produces BC, and at least one microorganism which does not produce BC and at least one microorganism which produces or is capable of producing at least one cellulytic agent.

In some embodiments, the co-culture may comprise at least one bacteria which produce BC, and at least one microorganism which produces or are capable of producing at least one cellulytic agent.

### Production of Bacterial Cellulose

The method for producing the BC may be any known fermentation method, such as for example batch fermentation, repeated batch fermentation, fed-batch fermentation, and continuous fermentation such as continuous agitated fermentation. The fermentation may be an open fermentation, preferably wherein the culture medium and/or the fermenter has not been sterilized prior to use. In some embodiments, the culture medium and/or the fermenter has not been sterilized prior to use.

Preferably, the fermentation is aerobic. However, there may be sections of the fermenter wherein the fermentation is microaerobic and/or anaerobic. The fermentation can further be conducted under static and/or agitated conditions. In one embodiment, the fermentation is static aerobic fermentation, such as without shaking. In another embodiment, the fermentation is agitated aerobic fermentation, such as with stirring and/or bubbling.

In some embodiments, the fermentation is performed at a temperature between 15 and 30° C., such as at 15° C., such as at 16° C., such as at 17° C., such as at 18° C., such as at 19° C., such as at 20° C., such as at 21° C., such as at 22° C., such as at 23° C., such as at 24° C., such as at 25° C., such as at 26° C., such as at 27° C., such as at 28° C., such as at 29° C., or 30° C., or such as between 18 and 28° C., such as between 20 and 26° C., such as between 22 and 24° C. In other embodiments, the fermentation is performed at room temperature. In other words, the step of incubating at least one cellulose-producing bacteria in a culture medium, and optionally, at least one other microorganism which does not produce cellulose, thereby obtaining BC in said culture medium, is performed at room temperature or at a temperature between 15 and 30° C., 15° C., such as at such as at 16° C., such as at 17° C., such as at 18° C., such as at 19° C., such as at 20° C., such as at 21° C., such as at 22° C., such as at 23° C., such as at 24° C., such as at 25° C., such as at 26° C., such as at 27° C., such as at 28° C., such as at 29° C., or 30° C., or such as between 18 and 28° C., such as between 20 and 26° C., such as between 22 and 24° C.

The fermentation may be performed until the desired amount of BC has been produced. In some embodiments, the fermentation is performed for a period of 2 to 10 days, such as 2 days, such as 3 days, such as 4 days, such as 5 days, such as 6 days, such as 7 days, such as 8 days, such as 9 days, such as 10 days. In other words, the step of incubating at least one cellulose-producing bacteria in a culture medium, and optionally, at least one other microorganism which does not produce cellulose, thereby obtaining BC in said culture medium, is performed for a period of is performed for a period of 2 to 10 days, such as 2 days, such as 3 days, such as 4 days, such as 5 days, such as 6 days, such as 7 days, such as 8 days, such as 9 days, such as 10 days, for example between 2 and 9 days, such as between 3 and 8 days, for example between 4 and 7 days, such as between 5 and 6 days.

### Produced and Recovered BC

The BC which is produced in step a) according to the method presented herein may be in any form, such as in the form of a pellicle or in the form of smaller pieces of BC. For example, in a static fermentation, the BC may be in the form of a pellicle, and in an agitated fermentation, the BC may be in the form of smaller pieces of BC. Said pellicle is usually formed at the surface of the culture medium, or at the interface between the surface and the culture medium. Thus, in some embodiments, the BC is recovered by removing it from the surface of the culture medium, or from the interface between surface and culture medium. In some embodiments, the BC pellicle has a thickness between 0.2 and 2 cm, such as 0.2 cm, such as 0.3 cm, such as 0.4 cm, such as 0.6 cm, such as 0.8 cm, such as 1.0 cm, such as 1.2 cm, such as 1.4 cm, such as 1.6 cm, such as 1.8 cm, such as 2.0 cm. In some embodiments, the BC pellicle has a thickness between 0.2 and 1.8 cm, such as between 0.4 and 1.6 cm, such as between 0.6 and 1.4 cm, such as between 0.8 and 1.2 cm, such as about 1.0 cm.

As stated before, BC has a more crystalline structure compared to plant cellulose. The crystallinity index (CI) may be used to describe the relative amount of crystalline material in cellulose. The CI of BC may for example be measure using XRD or infrared (IR) spectroscopy. The inventors have discovered that the CI of BC is dependent on the carbon source of the culture medium. For example, the inventors have shown that BC produced in glucose or sucrose has a higher CI than BC produced in glycerol or fructose.

The CI is correlated to other properties of the BC, such as for example its hydrophobicity and thermostability. Thermostability is the quality of a substance to resist irreversible change in its chemical or physical structure, such as by resisting decomposition or polymerization, at a relatively high temperature. Hydrophobicity is the physical property of a compound that allows it to be repelled by a mass of water. Both the hydrophobicity and the thermostability of BC are positively correlated with the CI.

In some embodiments, the BC, i.e. the BC produced in step a) and optionally recovered, has a CI of 0.05 to 3, such as 0.05, such as 0.1, such as 0.2, such as 0.3, such as 0.4, such as 0.5, such as 0.6, such as 0.7, such as 0.8, such as 0.9, such as 1.0, such as 1.1, such as 1.2, such as 1.3, such as 1.4, such as 1.5, such as 1.6, such as 1.7, such as 1.8 such as 1.9, such as 2.0, such as 2.2, such as 2.4, such as 2.6, such as 2.8, such as 3.0. In some embodiments, the CI is between 0.05 and 3, such as between 0.1 and 2.9, such as between 0.2 and 2.8, such as between 0.3 and 2.7, such as between 0.4 and 2.6, such as between 0.5 and 2.5, such as between 0.6 and 2.4, such as between 0.6 and 2.3, such as between 0.7 and 2.2, such as between 0.8 and 2.1, such as between 0.9 and 2.0, such as between 1.0 and 1.9, such as between 1.1 and 1.8, such as between 1.2 and 1.7, such as between 1.3 and 1.6, such as between 1.4 and 1.5, for example between 0.05 and 2, between 1.0 and 3.0, between 1.0 and 2.0.

In some embodiments, the BC, i.e. the BC produced in step a) and optionally recovered, has a CI of 0.05 to 8, for example between 0.1 and 7, such as between 0.5 and 6.5, such as between 1 and 6, such as 2.5, such as 3, such as 3.5, such as 4, such as 4.5, such as 5, such as 5.5, such as 6, or between 1 and 5, between 2 and 5, between 2 and 4, between 3 and 5, between 3 and 4, between 1 and 2, between 1 and 4, between 1 and 3, or between 2 and 3.

Further, the BC, i.e. the BC produced in step a) and optionally recovered, may have a specific degree of polymerization (DP). The DP can be calculated from the viscosity using methods known in the art and methods described herein. In some embodiments, the BC has a DP of 900 to 2500, such as 900, 1000, 1100, 1200, 1300, 1400, 1500, 1600, 1700, 1800, 1900, 2000, 2100, 2200, 2300, 2400, or 2500. In some embodiments, the BC has a DP of between 900 and 1200, between 1200 and 1500, between 1500 and 1800, between 1800 and 2100, or between 2100 and 2500.

### Sterilisation

The method for producing the BC may be comprise a step of sterilisation, preferably said step is performed before steps a to d. Most preferably, said step is performed before step c.

The step of sterilisation may comprise the process of killing, removing, inactivating or deactivating microorganisms, and can be a chemical, enzymatic and/or a physical process or treatment of a sample, suspension, BC suspension, culture medium, recovered BC, functionalized BC, and/or BC pellicle that likely contain microorganisms. In some embodiments, the step of sterilisation comprises a combination of processes and/or treatments, such as the combination of both chemical, enzymatic and physical treatment of a sample, suspension, BC suspension, culture medium, recovered BC, functionalized BC, and/or BC pellicle.

Sterilisation may be achieved through heat treatment, chemical treatment, enzymatic treatment, irradiation, physical treatment, pressure treatment such as high pressure, and/or filtration.

In some embodiments, sterilisation is a chemical sterilisation such as performed by chemical treatment. In some embodiments, sterilisation is a chemical sterilisation. In some embodiments, said chemical sterilisation comprises lowering the pH of a sample, suspension, BC suspension, culture medium, recovered BC, functionalized BC, and/or BC pellicle below pH 7. In some embodiments, sterilisation comprises enzymatic treatment of said sample, suspension, BC suspension, culture medium, recovered BC, functionalized BC, and/or BC pellicle, such as by addition of an enzyme. Thus, in some embodiments, said lowering of the pH of a sample, suspension, BC suspension, culture medium, recovered BC, functionalized BC, and/or BC pellicle below pH 7 is combined with enzymatic treatment of said sample, suspension, BC suspension, culture medium, recovered BC, functionalized BC, and/or BC pellicle, such as by addition of an enzyme to remove, digest, cleave, modify and/or transform cell debris, such as bacterial cell debris, possibly present in the sterilized sample, suspension, BC suspension, culture medium, recovered BC, functionalized BC, and/or BC pellicle.

In some embodiments, sterilisation is used to kill, remove, inactivate and/or deactivate the least one cellulose-producing bacterium. Said at least one cellulose-producing bacterium may be present, attached, entrapped or entangled in BC fiber network and/or BC pellicle of or within said sample, suspension, BC suspension, culture medium, recovered BC, functionalized BC, and/or BC pellicle.

### Treatment and Functionalization

After recovery, the obtained BC may be chemically treated, such as for example to sterilize or further functionalize it, or to further modify its properties in other ways.

The recovered BC may be sterilized using different methods and as detailed herein. In some embodiments, the recovered BC is contacted with a base, such as NaOH and/or NaCl₄, thereby sterilizing the BC. In some embodiments, the recovered BC is contacted with NaOH and/or NaCl₄, preferably NaOH, such as with 0.3 to 10% (w/w) NaOH and/or NaCl₄, such as 0.3%, such as 0.5%, such as 1%, such as 1.5%, such as 2%, such as 2.5%, such as 3%, such as 3.5%, such as 4%, such as 4.5%, such as 5%, such as 6%, such as 7%, such as 8%, such as 9% NaOH, thereby sterilizing the BC. In some embodiments, the recovered BC is contacted with NaOH and/or NaCl₄, preferably NaOH, such as with 0.5 to 9% NaOH and/or NaCl₄, such as 0.75 to 8%, such as 1 to 7%, such as 2 to 6%, such as 3 to 5%.

The recovered BC may further be functionalized. Functionalization of BC results in modification of the properties of said BC in a way which adds new functions, features, capabilities or properties to the BC by changing the surface chemistry of the BC. BC may for example be functionalized by introducing a functional group, such as a substituent or moiety to the surface of the BC material.

For the purpose of the present invention, the functionalization modifies the hydrophobicity of the BC. In particular, the functionalization of the BC provides a material with a higher hydrophobicity compared to a BC produced under the same conditions, which has not been functionalized. The hydrophobicity of the BC may be measured using methods known in the art, such as by measuring the water contact angle of the BC.

Thus, in some embodiments of the present invention, the functionalization of the recovered BC increases the hydrophobicity of said BC, wherein the hydrophobicity is defined as the water contact angle of the BC. In other words, functionalization of the BC increases the water contact angle of said BC.

In some embodiments, the water contact angle is increased from 45° to 150°, such as 50°, such as 60°, such as 70°, such as 80°, such as 90°, such as 100°, such as 110°, such as 120°, such as 130°, such as 140°, such as 45° to 75°, such as 45° to 120°, such as 45° to 60°, such as 45° to 95°. In some embodiments, the water contact angle is increased by 5° or more, such as by 10° or more, such as by 15° or more, such as by 20° or more, such as by 25° or more, such as by 30° or more, such as by 35° or more, such as by 40° or more, such as by 45° or more, such as by 50° or more, such as by 55° or more, such as by 60° or more, such as by 65° or more, such as by 70° or more, such as by 75° or more, such as by 80° or more, such as by 85° or more, such as by 90° or more, such as by 95° or more.

In some embodiments, the water contact angle is increased by 10 to 200%, such as 10%, such as 20%, such as 30%, such as 40%, such as 50%, such as 60%, such as 70%, such as 80%, such as 90%, such as 100%, such as 120%, such as 140%, such as 160%, such as 180%, such as 200%, such as by 20 to 95%, such as by 25 to 70%, such as by 25 to 60%, such as by 25 to 35%, such as by 30 to 50%, such as by 35 to 45%, such as by 40 to 60%, such 45 to 50%, such as by 50 to 60%, such as by 55 to 65%, such as by 60 to 80%, such as by 50 to 110%, such as by 30 to 70%, such as by 50 to 150%.

In some embodiments, the water contact angle is between 45° and 180°, such as between 45° and 170°, such as between 45° and 160°, such as between 45° and 150°, such as between 50° and 150°, such as between 60° and 150°, such as between 70° and 150°, such as between 70° and 150°, such as between 75° and 150°, such as between 80° and 150°, such as between 85° and 150°, such as between 90° and 150°, such as between 95° and 150°, such as between 100° and 145°, such as between 105° and 145°, such as between 110° and 150°, such as between 115° and 150°, such as between 120° and 150°, such as between 125° and 150°, such as between 130° and 150°, such as between 135° and 150°, such as between 140° and 150°, such as between 145° and 150°.

In some embodiments, the recovered BC is contacted with nitric acid or acetic anhydride, thereby functionalizing said BC, similar to what has been described above in relation to functionalization of the BC material used to produce the BC. In some embodiments, 1 to 80% of the accessible groups are modified, such as 1%, such as 5%, such as 10%, such as 20%, such as 30%, such as 40%, such as 50%, such as 60%; such as 70%, such as 80%, such as 5 to 60%, such as 10 to 40%, such as 15 to 30%, such as 5 to 15%. In some embodiments, at least 1% of the accessible groups are modified, such as at least 5%, at least 10%, at least 15%, at least 20%, at least 25%, at least 30%, at least 35%, at least 40%, at least 45%, at least 50%, or more.

In some embodiments, the concentration of nitric acid is 0.3 to 10% (v/v), such as 0.3%, such as 0.5%, such as 1%, such as 2%, such as 3%, such as 4%, such as 5%, such as 6%, such as 7%, such as 8%, such as 9%, such as 10%, such as 1 to 10%, such as 1 to 5%, such as 2 to 6%, such as 3 to 8%. In some embodiments, contacting the BC with nitric acid results in that at least some of the accessible hydroxyl groups of the BC are substituted with nitro groups. In some embodiments, 1 to 80% of the accessible hydroxyl groups are substituted with nitro groups, such as 1%, such as 10%, such as 20%, such as 30%, such as 40%, such as 50%, such as 60%, such as 70%, such as 80%, such as 10 to 30%, such as 10 to 40%, such as 15 to 60%, such as 5 to 20%, such as 40 to 70%. In some embodiments, at least 1% of the accessible hydroxyl groups are substituted with nitro groups, such as at least 5%, at least 10%, at least 15%, at least 20%, at least 25%, at least 30%, at least 35%, at least 40%, at least 45%, at least 50%, or more.

In some embodiments, the concentration of acetic anhydride is 1 to 25%, such as 1%, such as 2%, such as 3%, such as 4%, such as 5%, such as 6%, such as 7%, such as 8%, such as 9%, such as 10%, such as 15%, such as 20%, such as 25%, such as 5 to 15%, such as 1 to 10%, such as 20 to 25%, such as 3-12%. In some embodiments, contacting the BC with acetic anhydride results in that at least some of the accessible hydroxyl groups of the BC are substituted with acetate groups. In some embodiments, 1 to 80% of the accessible hydroxyl groups are acetylated, such as 10%, such as 20%, such as 30%, such as 40%, such as 50%, such as 60%, such as 70%, such as 10 to 30%, such as 10 to 40%, such as 15 to 60%, such as 5 to 20%, such as 40 to 70%. In some embodiments, at least 1% of the accessible hydroxyl groups are acetylated, such as at least 5%, at least 10%, at least 15%, at least 20%, at least 25%, at least 30%, at least 35%, at least 40%, at least 45%, at least 50%, or more.

To determine if the BC has been functionalized as defined in the present invention, IR can be used to measure the substitution or conversion of the hydroxyl groups into functional groups. The hydroxyl groups and the functionalized groups will appear at different wavenumbers on the infrared spectrum.

For example, attenuated total reflection, a type of infrared spectroscopy (AT-IR) can be used to measure the presence of hydroxyl groups as well as functionalized groups before and after treatment with the functionalizing agent. The hydroxyl groups will appear as a peak at a certain wavenumber, and the functionalized group will appear as a peak at another wavenumber. Thus, if some of the hydroxyl groups of the BC have been functionalized, the peak at the wavenumber of the hydroxyl group will decrease, while the peak at the wavenumber of the functionalized group will increase and/or emerge on the infrared spectrum of the BC after treatment with the functionalizing agent, compared the infrared spectrum of the BC before treatment with the functionalizing agent.

### Comminution

A suspension of BC, comprising ground BC, can be obtained using various methods known in the art, including but not limited to aqueous counter-collision, crushing, cutting, high-pressure homogenization, hydrolysis with bleach, an acid or an enzyme, mechanical shearing stress, trituration, ultrasound, wet grinding mills, or a combination thereof. The size of the obtained fiber clusters, i.e. the obtained fibril aggregates, of the ground BC may affect the properties of the BC suspension.

The inventors have observed that the comminution affects the properties of the BC suspension. For example, the method and degree of comminution determines whether or not the prepared BC suspension is suitable for use as a coating agent. The inventors have shown that comminution using a Masuko grinding stone generates a BC suspension which can be used to homogenously coat a material, while comminution using an IKA Turrax generates a BC suspension which agglomerates and is unable to homogenously coat a material. Furthermore, the inventors have found that using a Silverson homogenizer for comminution of pristine BC does not result in a BC suspension that is suitable for coating a material. Further to this, the inventors have also found that chemical modification of BC homogenized using a Silverson homogenizer results in a homogenous BC suspension.

The inventors have further discovered that the concentration of the BC solids after comminution affects the coating ability of the BC suspension. The inventors have shown that coating a material using a suspension with a solid content of 4.3% results in a non-homogenous coating due to agglomeration, while a solid content below 4.3% enables homogenous coating of the material. Any comminution which can lead to a solid content below 4.3%, such as 4.2% or less, for example 4.1% or less, such as 4.0% or less, such as between 1.6 and 2.3%, for example between 1.6% and 1.9% or between 2.1 and 2.3%, can thus advantageously be used.

In one embodiment of the present invention, the BC suspension is prepared by comminution of the recovered and/or the functionalized BC.

In some embodiments the recovered and/or functionalized BC is comminuted using at least one method selected from the group consisting of aqueous counter-collision, crushing, cutting, high-pressure homogenization, mechanical shearing stress, trituration, ultrasound, and/or wet grinding mills, such as at least two methods.

In some embodiments, the recovered and/or functionalized BC is comminuted using an IKA Turrax or a supermasscolloider, such as a Masuko grinding stone.

In some embodiments the recovered and/or functionalized BC is comminuted using a supermasscolloider, such as a Masuko grinding stone, at a speed of 500 to 3000 rpm, such as 500 rpm, such as 600 rpm, such as 700 rpm, such as 800 rpm, such as 900 rpm, such as 1000 rpm, such as 1200 rpm, such as 1300 rpm, such as 1400 rpm, such as 1500 rpm, such as 1600 rpm, such as 1700 rpm, such as 1800 rpm, such as 1900 rpm, such as 2000 rpm, such as 2100 rpm, such as 2200 rpm, such as 2300 rpm, such as 2400 rpm, such as 2500 rpm, such as 2600 rpm, such as 2700 rpm, such as 2800 rpm, such as 2900 rpm, such as 3000 rpm. In some embodiments, the speed is between 600 and 2800 rpm, such as between 700 and 2600 rpm, such as between 800 and 2400 rpm, such as between 900 and 2200 rpm, such as between 1000 and 2000 rpm, such as between 1200 and 1800 rpm, such as between 1400 and 1600 rpm.

In some embodiments, the recovered and/or functionalized BC is centrifuged before and/or after the comminution.

In some embodiments, the BC after is heated before, after and/or during the comminution. In some embodiments, the heating is performed at temperature between 30° C. to 200° C., such as 30° C., such as 35° C., such as 40° C., such as 45° C., such as 50° C., such as 55° C., such as 60° C., such as 65° C., such as 70° C., such as 75° C., such as 80° C., such as 85° C., such as 90° C., such as 95° C., such as 100° C., such as 125° C., such as 150° C., such as 175° C., such as 200° C. In some embodiments, the temperature is between 30° C. and 200° C., such as between 40° C. and 190° C., such as between 50° C. and 180° C., such as between 60° C. and 170° C., such as between 70° C. and 160° C., such as between 80° C. and 150° C., such as between 90° C. and 140° C., such as between 100° C. and 130° C., such as between 110° C. and 120° C., for example between 40° C. and 60° C., between 60° C. and 80° C., between 80° C. and 100° C., between 100° C. and 120° C., between 120° C. and 140° C., between 140° C. and 160° C., between 160° C. and 180° C., or between 180° C. and 200° C. In some embodiments, the heating is performed for 5 min to 10 h, such as 5 min, such as 10 min, such as 15 min, such as 30 min, such as 45 min, such as 1 h, such as 1.5 h, such as 2 h, such as 2.5 h, such as 3 h, such as 3.5 h, such as 4 h, such as 4.5 h, such as 5 h, such as 6 h, such as 7 h, such as 8 h, such as 9 h, such as 10 h, for example 10 min to 9 h, such as 20 min to 8 h, such as 30 min to 7 h, such as 1 h to 6 h, such as 2 h to 5 h, such as 3 h to 4 h.

In some embodiments, the BC is heated between 4 h and 5 h at a temperature between 40° C. and 50° C. In other embodiments, the BC is heated between 5 h and 6 h at a temperature between 40° C. and 50° C. In a preferred embodiment, the BC is heated for 5 h at 45° C.

The BC suspension generated by the comminution may be as defined in the section “Bacterial cellulose suspension”.

### EXAMPLES

### Example 1—Production of Bacterial Cellulose

### Objective

Production of BC using different carbon source in a static medium.

### Material and Methods

BC was synthesized using a co-culture (i.e. a consortium or a symbiotic culture) of bacteria, comprising two cellulose-producing bacteria and one type of yeast; Gluconobacter oxydans, Komagataeibacter rhaeticus and Hanseniaspora uvarum.
1. 100 mL of culture was mixed with 900 mL of HS (Hestrin & Schramm) medium with different carbon sources (see table 1). 1 L HS medium contained 5 g peptone, 5 g yeast extract, 1.15 g citrate, and 2.7 g disodium phosphate. The carbon sources used (per 1 l) were either 20 g/L sucrose, 20 g/L glucose, 20 g/L fructose or 30 g/L glycerol. All medium chemicals were supplied by Sigma Aldrich).
2. The co-culture was inoculated in HS medium, and was let standing at room temperature (RT) (22±1° C.) for approximately 7 days in static condition.
3. The BC was produced in the interface of water and air, and after the 7 days of culturing it was removed by hand and washed in distilled water.
4. The BC was washed with 1 M NaOH for 60 min, and with 0.1 M HCl for 60 min to remove the alkalinity. Afterwards, it was washed thoroughly with distilled water.
5. Determination of degree of polymerization: Degree of Polymerization (DP) was calculated from the viscosity according to SCAN-C 15:62 and the following relationship:

$$D {P}^{0.905} = 0.75 \cdot n$$

where n is viscosity and DP is Degree of Polymerization. Viscosity was measured according to ISO 5351:2010.
6. Determination of crystallinity: The crystallinity index (CI) was determined with Fourier Transform Infrared spectroscopy (FT-IR) according to the method disclosed by O'Connor et al. (1958). The crystallinity index (CI) was determined by calculating the peak ratio at 1430 and 898 cm⁻¹.

### Results

Various parameters of the BC produced in HS medium with different carbon sources can be seen in table 1.

TABLE 1
DP and crystallinity of BC grown and obtained from
HS medium using different carbon sources.

| Carbon source in | | Viscosity, | Crystallinity | Crystallinity |
|---|---|---|---|---|
| HS medium | DP | ml/g | index | % |
| 3% Glycerol | 934 | 650 | 0.013 | 1.2 |
| 2% Fructose | 1045 | 720 | 0.078 | 7.3 |
| 2% Sucrose | 1737 | 1140 | 1.31 | 56-94 |
| 2% Glucose | 1974 | 1280 | 1.24 | 55 |
| 10% sucrose | 1500-2000 | 977-1300 | 2.1-6.4 | 60-94 |

BC obtained using glucose and sucrose as carbon source in HS medium was shown to have a higher DP and crystallinity compared to that obtained using glycerol and fructose (Table 1).

We hypothesized that a high CI be advantageous in the formulation of a BC suspension for barrier coating. Thus, we used BC produced in sucrose-containing medium for the following experiments within the concentration range of 2 to 10%.

### Example 2—Treatment of the Recovered BC

### Objective

Sterilization and functionalization of the BC.

### Material and Methods

1. The BC obtained from the static culture, grown in a static condition in 2% sucrose HS medium, was washed in NaOH solution with different concentrations (from 0.1-25% (w/v)).
2. Each washing step was carried out for 24 h.
3. Optional: Treatment using different concentrations of alcalase to remove potentially attached or entrapped proteins from lysed bacteria.
4. The BC obtained after washing or alcalase treatment was then washed with distilled water and placed on the top of a 2% sucrose HS medium-agar plate for 24 h to check if any microorganisms grew (i.e. to control its sterility).
5. The BC from the best-performing NaOH washing step was functionalized by immersing it in nitric acid (HNO₃) solutions with various concentrations, whereby some of the accessible hydroxyl (OH) groups of the cellulose fibers were substituted with nitro groups.
6. The functionalized BC was washed extensively in distilled water to completely remove the reagents.
7. AT-IR was used to evaluate the functionalization step. The nitro:OH IR stretch was measured, wherein the nitro groups emerged at a wavenumber of 1515-1560 and 1345-1385 cm⁻¹, whereas the OH groups emerged at 2500-3300 cm⁻¹.

### Results

The results of the sterilization of the produced BC in different concentrations of NaOH and the possible additional treatment with different concentrations of alcalase can be seen in table 2.

TABLE 2
The physical appearance and microorganism growth after the sterilization
process using different concentrations of NaOH. NA: not applicable.

| | Physical | Microbial | Cellulose |
|---|---|---|---|
| NaOH | appearances | growth after | crystallinity |
| (% w/v in water) | after treatment | treatment | peak at 1430 |
| 0.1 | yellow | + | NA |
| 0.25 | yellow | + | NA |
| 0.5 | white | − | NA |
| 1 | white | − | No reproducible |
| | | | visible peak |
| 5 | brown, floating | − | NA |
| 10 | brown, floating | − | NA |
| 20 | yellowish, floating, | − | NA |
| | burnt | | |
| 25 | yellowish, floating, | − | NA |
| | burnt | | |
| 1% NaOH + | White | ′− | No reproducible |
| 0.125 g/L alcalase | | | visible peak |
| 1% NaOH + | White | ′− | Reproducible |
| 0.25 g/L alcalase | | | visible peak |
| 1% NaOH + | White | ′− | Reproducible |
| 0.5 g/L alcalase | | | visible peak |
| 1% NaOH + | White | ′− | Reproducible |
| 1 g/L alcalase | | | visible peak |

It was shown that a concentration of 0.5-1% NaOH was effective in sterilizing the BC (Table 2). At this concentration the colour and the quality of the BC was maintained. However, crystalline cellulose could not be reproducibly detected with Fourier-transform infrared spectroscopy (FTIR) due to a huge protein-related amine (NH₂) wave in the spectra, because it makes it difficult to measure the CI. Thus, addition of a minimum of 0.25 g/L alcalase to the sample cleaned the BC enough from protein to enable measurement and quantification of the CI.

The results of the functionalization of the sterilized BC using various concentrations of HNO₃ can be seen in table 3.

TABLE 3
The physical appearance of BC and emergence of a nitro peak
replacing the OH peak after functionalization using a HNO₃ solution.

| % HNO₃ | | Nitro peak |
|---|---|---|
| (v/v in water) | Physical appearances | (Nitro:OH peak ratio) |
| 0.1 | White | No nitro peak |
| 0.25 | White | No nitro peak |
| 0.5 | White | No nitro peak |
| 1 | Transparent | Nitro peak (3) |

It was shown that using a minimum of 1% HNO₃ during functionalization enabled functionalization of the accessible OH groups of BC to nitro groups, while still maintaining the transparency of the material (Table 3).

### Example 3—Preparation of a BC Suspension by Comminution of the Recovered BC

### Objective

Show how various comminution methods and parameters affect the properties of the BC suspension.

### Material and Methods

The BC which had been sterilized and functionalized was comminuted with different methods; a food disintegrator (IKA Turrax), a combination of food disintegrator and ultrasonication (Soniprep 150, ultrasonic disintegrator), a combination of food disintegrator, ultrasonication and heat (ultrasonic bath, Branson CPX 800 H-E), ultra-fine friction grinding machine supermasscolloider IV (Masuko MKZA10 series), or a homogenizer (Silverson L5M).

Resulting suspension particles size distribution was measured using a particle size analyzer (Malvern Mastersizer 2000) or laser diffraction (LD, MIE model).

To decrease the water content and increase the solid content of the obtained suspension, pretreatment by centrifugation (Sorvall RC6) was conducted at different time and speed.

Procedure for chemical modification of BC to produce BC of low CI:
a) BC pellicles was first centrifuged at 4000 rpm to up-concentrate it as much as possible to have an approximate solid content of 2%.
b) The BC pellicles were randomly cut into smaller fragments by blending.
c) 0.3 g of cut BC pellicles was mixed with 0.3 g of NaBr/NaClO4 and mixed at 80 C for 2 hours.
d) Modified BC was washed extensively with 5 L DIW, in which the BC was retained using 38 μm sieve. The washing was performed at least 3 times.
e) Then, the BC was homogenized using Silverson homogenizer at 10,000 rpm for 30 minutes until no flocculating fibers can be seen with naked eye.
f) Crystallinity % was determined in the same manner as previously described.

### Results

The fiber cluster size and solid content of the BC suspensions obtained after various comminution methods can be seen in table 4.1 and table 4.2, that also includes median particle/fiber cluster size and crystallinity %.

TABLE 4.1
Particle or fiber cluster size and solid content of the BC suspension
after different methods of communication.

| | Particle/fiber cluster | Solid |
|---|---|---|
| Comminution method | size, μm | content, % |
| A. Ultrasonication, 10 min at 50° C. | undispersed | — |
| B. Comminution using IKA Turrax | 100-1000 | 1.27 |
| for 13 mins | | |
| 10 mins at 9500 rpm | | |
| 1 min at 13500 rpm | | |
| 1 min at 20500 rpm | | |
| 1 min at 24000 rpm | | |
| C. Comminution using IKA Turrax | 400-700 | 1.1 |
| for 13 mins | | |
| 10 mins at 9500 rpm | | |
| 1 min at 13500 rpm | | |
| 1 min at 20500 rpm | | |
| 1 min at 24000 rpm | | |
| 10 min ultrasonication for 10 mins | | |
| D. Comminution using Masuko | 100-400 | 1.6 |
| grinding stone (static and running) | | |
| at 1500 rpm | | |
| E. Comminution using Masuko | 100-400 | 2.3 |
| grinding stone (static and running) | | |
| at 1500 rpm | | |
| Heating at 45° C. for 5 H | | |
| Centrifugation at 4000 rpm at 20° C. | 10-500 | 4.3 |
| F. Comminution using Masuko | | |
| grinding stone (static and running) | | |
| at 1500 rpm | | |
| Heating at 45° C. for 5 H | | |

TABLE 4.2
Particle or fiber cluster size and solid content of the BC suspension after
different methods of comminution. *chemical modification induces
OH group formation, which decreases the crystallinity %(/CI)

| | Particle/ | Particle/ | | |
|---|---|---|---|---|
| | fiber | fiber | | |
| | cluster | cluster | Solid | |
| Comminution | size, | median | content, | Crystallinity, |
| method | μm | size, μm | % | % |
| A. Ultrasonication, | un- | un- | — | <94 |
| 10 min at 50° C. | dispersed | dispersed | | |
| B. Comminution | 75-1240 | 150-400 | 1.27 | <94 |
| using IKA Turrax | | | | |
| for 13 mins | | | | |
| 10 mins at 9500 rpm | | | | |
| 1 min at 13500 rpm | | | | |
| 1 min at 20500 rpm | | | | |
| 1 min at 24000 rpm | | | | |
| C. Comminution | 75-1300 | 150-400 | 1.1 | <94 |
| using IKA Turrax | | | | |
| for 13 mins | | | | |
| 10 mins at 9500 rpm | | | | |
| 1 min at 13500 rpm | | | | |
| 1 min at 20500 rpm | | | | |
| 1 min at 24000 rpm | | | | |
| 10 min ultrasonication | | | | |
| for 10 mins | | | | |
| D. Comminution | 5-1240 | 75-100 | 1.6 | <94 |
| using Masuko | | | | |
| grinding stone (static | | | | |
| and running) at 1500 rpm | | | | |
| E. Comminution | 5-1240 | 75-100 | 2.3 | <94 |
| using Masuko grinding | | | | |
| stone (static and running) | | | | |
| at 1500 rpm | | | | |
| Heating at 45° C. for 5 H | | | | |
| Centrifugation at 4000 | 5-1240 | 75-100 | 4.3 | <94 |
| rpm at 20° C. | | | | |
| F. Comminution | | | | |
| using Masuko grinding | | | | |
| stone (static and running) | | | | |
| at 1500 rpm | | | | |
| Heating at 45° C. for 5 H | | | | |
| Centrifugation at | 75-1300 | 150-400 | 0.3-1 | <94 |
| 4000 rpm at 20 C. | | | | |
| G. Homogenization | | | | |
| using Silverson | | | | |
| homogenizer at 10,000 | | | | |
| rpm for 30 mins | | | | |
| Centrifugation at | 1-200 | 50-100 | 0.3-1 | <50 |
| 4000 rpm at 20 C. | | | | |
| H. Chemical modification | | | | |
| using NaBr/NaClO4 for | | | | |
| 2 H Homogenization | | | | |
| using Silverson | | | | |
| homogenizer at 10,000 | | | | |
| rpm for 30 mins* | | | | |

Comminution with ultrasonication (A), IKA turrax (B, C) or Silverson homogenizer (G) did not give a stable BC suspension, as there was still a big lump which could not be spread on a surface of a material evenly (Table 4.1 and Table 4.2). Comminution using the Masuko supermasscolloider (D, E, F) or the combination of chemical modification and Silverson homogenizer (H) gave a more stable suspension with homogenously dispersed suspensions particles, that could be evenly distributed on the surface of a material. However, chemical modification seems to result in a BC suspension of lower crystallinity (H) that retains more water.

### Example 4—Characterization of the BC Suspensions

### Objective

Characterization of the BC suspensions.

### Material and Methods

The viscosity (Pa·s) versus the shear rate (s⁻¹) of the suspension was determined with a rheometer (DH-2, TA instruments EQ199) at 25° C., at a shear rate between 0.1-1000 s⁻¹.

The fiber cluster size was determined using MORFI analysis with MORFI LB01 system or laser diffraction (LD) with LD MIE model.

### Results

The viscosities at different shear rates of the BC suspensions obtained with various comminution methods can be seen in table 5.

TABLE 5
The viscosity of BC suspensions which were comminuted using different
methods (Pa · s) at different shear rates (s⁻¹).

| | Viscosity, Pa · s | | | | | |
|---|---|---|---|---|---|---|
| Comminution method | 0.001/s | 0.01/s | 0.1/s | 1/s | 10/s | 100/s |
| A. Ultrasonication, 10 min at 50° C. | — | — | — | — | — | — |
| B. Comminution using IKA | 2000 | 900 | 90 | 10 | 1 | 0.1 |
| Turrax for 13 mins | | | | | | |
| 10 mins at 9500 rpm | | | | | | |
| 1 min at 13500 rpm | | | | | | |
| 1 min at 20500 rpm | | | | | | |
| 1 min at 24000 rpm | | | | | | |
| C. Comminution using IKA | 5000 | 1000 | 100 | 15 | 3 | 0.3 |
| Turrax for 13 mins | | | | | | |
| 10 mins at 9500 rpm | | | | | | |
| 1 min at 13500 rpm | | | | | | |
| 1 min at 20500 rpm | | | | | | |
| 1 min at 24000 rpm | | | | | | |
| 10 min ultrasonication for 10 mins | | | | | | |
| D. Comminution using Masuko | — | — | 1400 | 215 | 31 | 5.1 |
| grinding stone (static and | | | | | | |
| running) at 1500 rpm | | | | | | |
| E. Comminution using Masuko | — | — | 356 | 52 | 4.8 | 0.52 |
| grinding stone (static and | | | | | | |
| running) at 1500 rpm | | | | | | |
| Heating at 45° C. for 5 H | | | | | | |
| Centrifugation at 4000 rpm at 20° C. | — | — | 7100 | 880 | 101 | 7.7 |
| F. Comminution using Masuko | | | | | | |
| grinding stone (static and | | | | | | |
| running) at 1500 rpm | | | | | | |
| Heating at 45° C. for 5 H | | | | | | |
| Centrifugation at 4000 rpm at 20 C. | — | — | 0.1 | 0.05 | 0.01 | 0.005 |
| G. Homogenization using | | | | | | |
| Silverson homogenizer at | | | | | | |
| 10,000 rpm for 30 mins | | | | | | |
| Centrifugation at 4000 rpm at 20 C. | — | — | 5 | 1 | 0.5 | 0.1 |
| H. Chemical modification using | | | | | | |
| NaBr/NaClO4 for 2 H | | | | | | |
| Homogenization using Silverson | | | | | | |
| homogenizer at 10,000 rpm for | | | | | | |
| 30 mins* | | | | | | |

*chemical modification induces OH group formation, which decreases the crystallinity % (/CI)

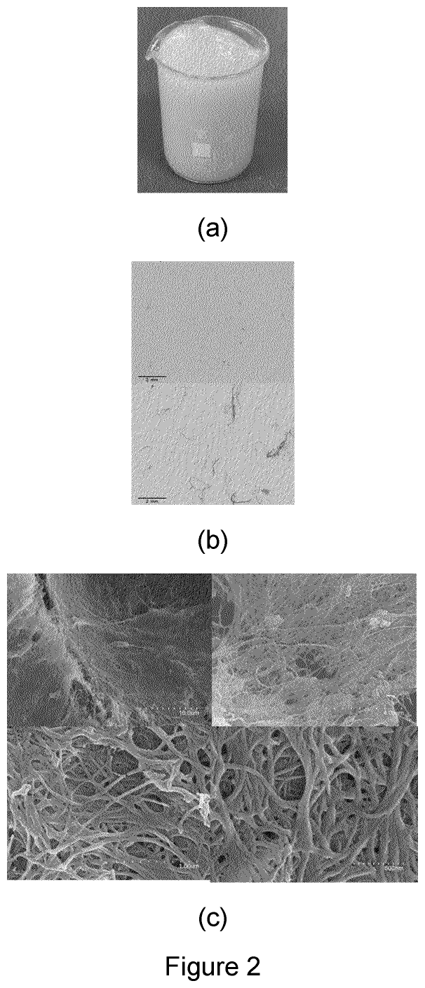

The BC suspensions best suited for coating were suspensions C-F and H (Table 5, FIG. 2). Suspension B and G could not be used, due to the fact that a big clump of clusters was found in said suspension. Thus, further characterization using MORFI analysis was carried out of suspensions C-F, which will further be used for paper coating, and H.

The fiber cluster size and surface area of the BC suspensions C-F and H obtained with various comminution methods can be seen in table 6.

TABLE 6
The fiber cluster size and surface area of the BC suspensions
which had been comminuted using different methods.

| | Fiber size | | | |
|---|---|---|---|---|
| | Length, | Width, | Diameter, | Surface |
| Method | μm | μm | μm | area, μm² |
| Suspension C | 738 ± 29 | 34.7 ± 0.2 | 30 | 98,300 |
| Suspension D, E, F | 416 ± 9 | 36.5 ± 1.5 | 30 | 57,600 |
| Suspension H | 1-200 | 1-200 | 30 | 270,300 |

Chemical modification and homogenization using Silverson homogenizer (Suspension H) results in the smallest fiber clusters and most dispersed BC suspension compared to using the food disintegrator (Suspension C) or supermasscolloider (Suspensions D, E, F) (Table 6 and Tables 4.1 and 4.2). However, chemical modification of the fibers results in lower crystallinity. Suspension H is more dispersed when homogenized due to more exposed OH groups (Table 4.2), which has a tendency to absorb more water.

Therefore using a supermasscolloider (Suspension D, E, F) is the best method for comminution of BC, as it resulted in small fiber clusters and a nicely dispersed BC suspension.

### Example 5—Application of the BC Suspension

### Objective

To use the BC suspensions for emulsification (example 5.1), particle stabilization (example 5.2) and coating (example 5.3) applications.

### 5.1. BC Suspension as Emulsifier (Oil in Water)

### Material and Methods

We used liquid parrafin (parraffinum liquidum) as the oil part of the emulsion. Suspensions of other natural polymers were used for comparison: plant microfibrillated cellulose (plant MFC, extracted from wood), carboxymethyl cellulose (CMC, MW 90,000), xanthan gum (XG, from Xanthomonas campestris).

### Procedure:

a) The BC suspension or other natural polymer suspensions were diluted to 1 (w/v) % with DIW and added to a tube to achieve the range of concentrations 0.1-1 (w/v) % of polymer.
b) 2 mL of the liquid paraffin was added to the each tube.
c) The mixture was mixed with Silverson homogenizer at 25000 rpm for at least 4 minutes per sample, without any further addition of emulsifier.
d) Pictures of the oil-water separation were taken after overnight incubation at room temperature (RT).

### Results

TABLE 7
Emulsification properties of BC suspension D or E or other
polymer to liquid parrafin. Minus (−) sign shows no
emulsification, plus (+) sign shows stable
emulsification in the concentration stated on the first column.

| Final | Final | | | | |
|---|---|---|---|---|---|
| concentration | concentration | BC | | | |
| of polymer, | of liquid | Suspension | Plant | | |
| (w/v) % | parrafin, (v/v) % | D or E | MFC | CMC | XG |
| 0.1 | 20 | − | − | − | − |
| 0.2 | 20 | − | − | − | − |
| 0.3 | 20 | − | − | − | − |
| 0.4 | 20 | − | − | − | − |
| 0.5 | 20 | − | − | − | − |
| 0.6 | 20 | − | − | − | − |
| 0.7 | 20 | − | − | − | − |
| 0.8 | 20 | − | − | − | − |
| 0.9 | 20 | + | + | − | − |
| 1.0 | 20 | + | + | − | − |

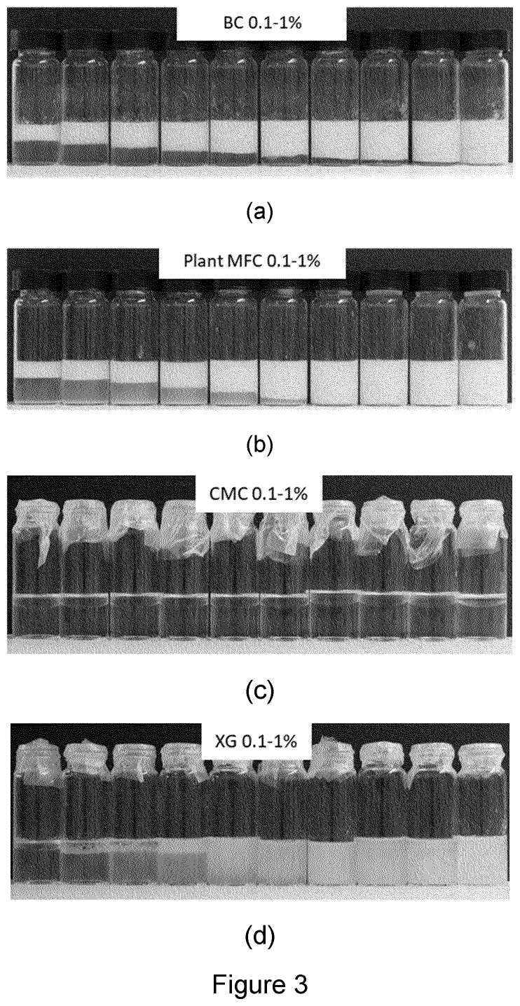

It was shown that BC suspension D and/or E can be used as emulsification agent (emulsifier) at a concentration of minimum 0.9 (w/v) %, similar to plant MFC (Table 7, FIG. 3 a-b). CMC or XG did not show any emulsification capability in the range of polymer concentrations tested (Table 7, FIG. 3 c-d).

### 5.2. BC Suspension as Particle Stabilizator in Suspension

### Material and Methods

In this test, we used different types of plant MFC (Plant MFC1 and Plant MFC2) as the other natural polymers CMC and XG could not be used as emulsifier as shown in Table 7 and FIG. 3 c-d. Activated charcoal was used as particles to be stabilised in suspensions.

### Procedure:

10 ml of 0.1, 0.5 and 1 (w/v) % of BC or other polymer suspensions as comparison in each tube were prepared.
0.5 mg of activated charcoal (particle size of 1.5 mm) were mixed with the polymer suspensions in the tube using a simple mixer for at least 4 minutes.
Pictures were taken after overnight incubation at RT.

### Results

TABLE 8
Particle stabilization properties of BC suspensions D or E or other
plant MFC of activated charcoal. Minus (−) sign shows no
particle stabilization, plus (+) sign shows stable particle
in suspension in the concentration stated on the first column.

| | BC | | |
|---|---|---|---|
| Concentration of | Suspension | | |
| polymer, w/v % | D or E | Plant MFC1 | Plant MFC2 |
| 0.1 | − | − | − |
| 0.5 | + | − | − |
| 1.0 | + | + | + |

It was shown that BC suspension D and/or E can be used as particle stabilizer (particle stabilizator) in suspensions with polymer concentrations of minimum 0.5 (w/v) % (Table 8, FIG. 4 A), while both plant MFCs (Plant MFC1 and Plant MFC2) need twice (2×) the concentration (1.0 (w/v) %) to give a stable particle suspension (FIG. 4 B-C).

### 5.3. BC Suspension as Coating Agent

### Material and Methods

We used three types of papers for coating; paper A (MONDI, 60 g/m²), paper B (MG, 50 g/m²) and paper C (NATURA, 120 g/m²).

The coating was conducted using a TQC Automatic Film Applicator Standard AB4400 with a perforated heated vacuum bed (TQC sheen). Before application, the substrate was held securely by clamps and then, the coating formulation was spread as a consistent and reproducible film across the surface with the aid of an applicator. As a function of the desired thickness, different stainless-steel film applicators were used to apply the coating. The wet coating thicknesses evaluated were 30, 50 and 100 μm.

We also evaluated the use of additive or plasticizer to the suspension, such as glycerol, polyvinyl alcohol and cationic starch (Sigma Aldrich).

BC Suspension Supplemented with Glycerol

Glycerol was selected as an additive in order to improve the flexibility of the coating and potentially the homogeneity of the dry coating. Two different ratios of glycerol were evaluated based on previous knowledge: 5% and 10% of the total weight of the formulation. Two batches of 100 grams each were prepared by the addition of 5 and 10 grams of glycerol to 95 and 90 grams of BC suspension, respectively. The coating preparation procedure was as follows; the proper amount of glycerol was added in the selected ratios to the BC suspensions, and all was homogenised with an Ultraturrax IKA T25 for 15 minutes at between 8000-9000 rpm and at ambient conditions. After preparation, the solution was applied for paper coating.

BC Suspension Supplemented with Poly(Vinyl Alcohol) (PVOH)

Polyvinyl alcohol (PVOH) was selected as an additive for the improvement of the flexibility and the homogeneity of the BC coatings. PVOH has been shown to increase the tensile strength of paper substrates, and to improve the barrier properties of the coating.

Two different ratios of PVOH were evaluated: 5 and 10% of the total weight of PVOH in solid form. Dissolution of PVOH in water was done before adding it to the suspension. The solubility of PVOH in water is low, as the highest content of PVOH which can be dissolved in water is a 20%. Thus, the PVOH solution was prepared by the addition of 20 grams of PVOH to 80 grams of water. The solution was left under stirring at 100° C. overnight.

Two batches of 100 gram each were prepared by the addition of 5 and 10 grams of 20% PVOH dissolution to 95 and 90 grams of BC suspension, respectively. The coating preparation procedure was as follows; the proper amount of PVOH solution was added in the selected ratios to the BC suspensions, and all was homogenised with an Ultraturrax IKA T25 for 20 minutes between 9000-10000 rpm at ambient conditions. After preparation, the solution was applied for paper coating. Since it was not possible to obtain a BC suspension with a high PVOH content, two alternatives with this additive were tested, and the application of two layers onto the paper was also evaluated, wherein the first layer consisted of a 2% PVOH suspension (4 μm coating), and the second layer consisted of BC suspension (100 μm coating).

BC Suspension Supplemented with Cationic Starch

Starch was selected as an additive for the BC suspension coating formulation. Two different ratios of starch were evaluated: 5 and 10% of the total weight of the formulation. Two batches of 100 gram each were prepared by the addition of 5 and 10 grams of cationic starch to 95 and 90 grams of BC suspension, respectively. The coating preparation procedure was as follows; the proper amount of cationic starch was added in the selected ratios to the BC suspension and homogenised with an Ultraturrax IKA T25 for 15 minutes between 7000-8000 rpm at ambient conditions. After preparation, the solution was applied for paper coating.

### Grammage

The grammage of a handsheet paper is defined as the mass per unit area in g/m². The ISO 536 standard 2 method has been used for basis weight determination (ISO 536:2012). Five handsheets (area of each sheet was 200 cm) were weighed on a balance to the closest 0.01 g, and the total mass of the 2 handsheets was recorded. Grammage was then calculated from the following equation:

$$Basis weight = W / 5 \cdot 27.56$$

wherein W is weight per kg.

### Water Absorption (COBB Method)

The water absorption by the COBB method was evaluated following the standard UNE EN 20535:1996, in which the water absorption capacity of the paper network is evaluated by weighing the sample before and after placing it under a set volume of water (100 milliliters of water), for a specified period of time (60 sec.).

### Smoothness (Bendtsen Method)

The determination of smoothness and porosity of the paper is important to predict the behaviour of the coating, and its application method on the paper.

First, smoothness was evaluated following the standard ISO 8791-2:2013: Paper and board. Determination of roughness/smoothness (air leak methods)—Part 2: Bendtsen method. The Bendtsen method measures the air flow rate and the smoothness is expressed in milliliters of air per minute (ml/min). Smoothness evaluates the irregularities that disturb the ideal surface of the plane, a property which is related to numerous parameters, such as paper composition, and the different steps of the paper production process.

### Porosity (Bendtsen Method)

Finally, the porosity was evaluated following the standard ISO 5636-3:2013: Paper and board-Determination of air permeance (medium range)—Part 3: Bendtsen method. Bendtsen roughness is measured by clamping the test piece between a flat glass plate and a circular metal head and measuring the rate of airflow in ml/minute between the paper and head.

### OTR (Oxygen Transmission Rate)

The oxygen permeability assessment, expressed as an oxygen transmission rate (OTR), was conducted in an OX-TRAN 2/21 and OX-TRAN 2/22 (Mocon, Inc., Minneapolis, MN), in accordance with ASTM D3985-17. The results were expressed in the form of permeability with the correction of the sample thickness in each case.

### WVTR (Water Vapor Transmission Rate)

The water vapor permeability assessment, expressed as water vapor transmission rate (WVTR), was conducted on a PERMATRAN-W Model 3/33 (Mocon, Inc., Minneapolis, MN) equipment. The WVTR was measured at 23° C. and 50% relative humidity (RH). Measure range from 0.005 to 2000 g/m² day.

### Results

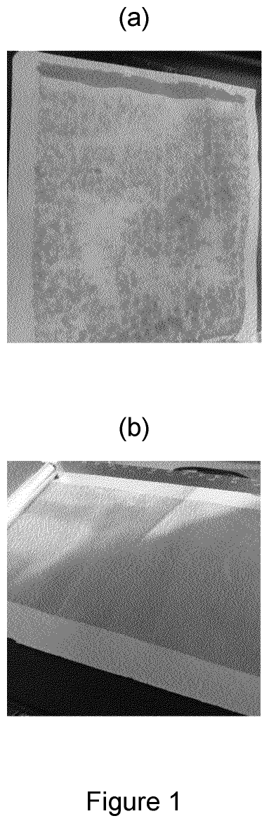

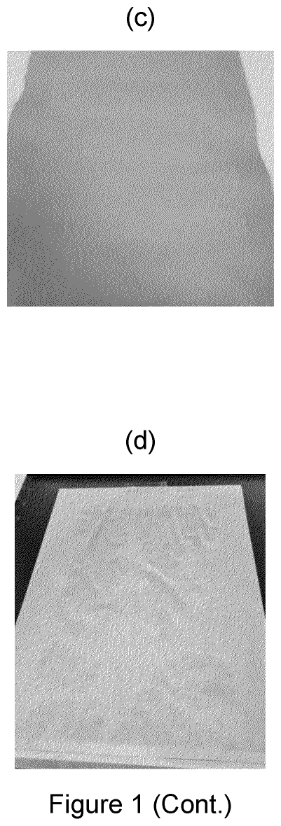

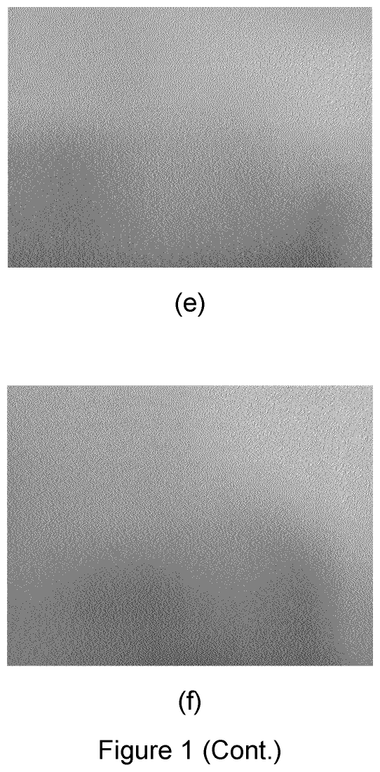

The BC suspensions C-F were used to coat paper. The results can be seen in table 7 and FIG. 1.

TABLE 9
The physical appearance of the paper coated with BC
suspensions C-F. See also FIG. 1.

| | Solid | |
|---|---|---|
| Samples | content, % | Physical appearance and remarks |
| Suspension C | 1.1 | Non-homogenous coating due to |
| | | agglomeration, non-homogenous |
| | | suspension dispersion. |
| Suspension D-E | 1.6 and 2.3 | Homogenous coating |
| Suspension F | 4.3 | Non-homogenous coating due to |
| | | agglomeration of fiber due |
| | | to loss of water. |

From the results seen in table 9 and FIG. 1, it was determined that the BC suspensions best suited for further formulation were suspension D and E. BC suspension C and F could not be used for preparation of an even coating due to the presence of big fiber clusters which formed ‘coating islands’. Suspension C contained big lump as a result of an improper comminution, while suspension F contained fibers aggregates due to a loss of water, resulting in flocculation of the fibers.

Thus, BC suspensions D and E were shown to be well suited for paper coating purposes, as they could be spread evenly and dry easily under operational parameters, which is a highly important feature when coating paper.

Thus, various coating properties of BC suspensions D and E were tested for the coating of different types of papers (table 10-14).

TABLE 10
The effect of different BCs coating deposition thickness to paper
A's grammage, smoothness, porosity and water absorption.

| | | Wet | | | | |
|---|---|---|---|---|---|---|
| | Solid | coating | | | | Water |
| Suspension | content, | thickness | Grammage | Smoothness | Porosity | absorption |
| formula | % | (μm) | (g/m²) | (mL/min) | (mL/min) | (g/m²) |
| Paper A | — | — | 59.9 ± 0.1 | 311.6 ± 9.6 | 172.8 ± 1.6 | 24.9 ± 1.6 |
| Paper A Susp D | 1.6 | 30 | 61.0 ± 0.5 | 344.5 ± 10.6 | 145.0 ± 5.7 | 18.4 ± 0.7 |
| Paper A Susp D | 1.6 | 50 | 61.1 ± 0.9 | 346.2 ± 25.9 | 141.0 ± 14.1 | 19.2 ± 1.1 |
| Paper A Susp D | 1.6 | 100 | 62.4 ± 0.9 | 366.5 ± 29.1 | 82.0 ± 1.4 | 19.5 ± 0.9 |
| PaperA Susp E | 2.3 | 30 | 62.4 ± 0.5 | 332.3 ± 12.5 | 117.0 ± 4.2 | 18.3 ± 0.4 |
| PaperA Susp E | 2.3 | 50 | 62.6 ± 0.3 | 359.7 ± 16.8 | 108.0 ± 3.5 | 18.1 ± 0.6 |
| PaperA Susp E | 2.3 | 100 | 63.6 ± 0.6 | 344.5 ± 42.4 | 67.0 ± 5.7 | 18.2 ± 0.7 |

From table 10 it was shown that:
Paper grammage increases by depositing thicker BC suspension which is related to the increase of the amount BC suspension deposited.
Porosity of the paper decreases when the paper is coated with a BC suspension, which is related to an increase of the barrier to oxygen.
Paper smoothness increases after BC suspension coating in general.
Water absorption decreases when the paper is coated with BC suspension, especially BC suspension E.
Suspension E with a solid content of 2.3% was performing better as a barrier coating than suspension D with a solid content of 1.6%. However, as shown earlier, higher solid content than 4% showed uneven coating due to agglomeration. Thus the range of 1.6-2.3% is preferable for use for further formulation.

Thus, various coating properties of BC suspension D with different additives were tested for the coating of different types of papers (table 11).

TABLE 11
The effect of different BCs coating formulation to paper
B's grammage, smoothness, porosity and water absorption.

| | | Wet | | | | |
|---|---|---|---|---|---|---|
| | Solid | coating | | | | Water |
| Suspension | content, | thickness, | Grammage | Smoothness | Porosity | absorption |
| formula | % | (μm) | (g/m²) | (mL/min) | (mL/min) | (g/m²) |
| Paper B | — | — | 51 ± 0.2 | 772 ± 314 | 193 ± 15 | 17.3 ± 0.1 |
| Paper B 100% | 1.6 | 100 | 52.5 ± 0.2 | 366.5 ± 29.1 | 82 ± 1.4 | 19.5 ± 0.9 |
| susp D | | | | | | |
| Paper B 5% | 6.5 | 100 | 50.6 ± 0.2 | 441 ± 102 | 178 ± 6 | 18.7 ± 0.9 |
| starch 95% | | | | | | |
| susp D | | | | | | |
| Paper B 5% | 6.5 | 100 | 50.9 ± 0.1 | 391 ± 54 | 197 ± 9 | 18.9 ± 0.3 |
| glycerol 95% | | | | | | |
| susp D | | | | | | |
| Paper B 5% | 6.5 | 100 | 51.1 ± 0.4 | 1771 ± 542 | 145 ± 9 | 17.4 ± 0.8 |
| PVOH 95% | | | | | | |
| susp D | | | | | | |
| Paper B 1^{st} | 1^{st} layer: 1.6, | 104 | 53.9 ± 0.4 | 878 ± 173 | 5 ± 2 | 17.8 ± 0.2 |
| layer: 4 μm | 2^{nd} layer: 2 | | | | | |
| 2% PVOH | | | | | | |
| 2^{nd} layer: | | | | | | |
| 100% susp D | | | | | | |

From table 11:
There was no improvement in porosity from the addition of glycerol, starch or PVOH to the BC suspension for coating application, unless the PVOH was used as a secondary layer before coating with suspension D. This effect is due to the hydrophilicity of PVOH.
There was no improvement in smoothness from the addition of glycerol, starch or PVOH to the BC suspension for coating application. Using only BC suspension D for coating was superior compared to using BC suspension D supplemented with glycerol, starch or PVOH.

The use of a multi-layer coating, i.e. a coating with one layer of pure BC suspension and further layers with other compounds, such as mineral suspension and other cellulose derivatives, was also explored. The oxygen transmission rate was measured for both the single-later BC coating, and for the multi-layer coatings (table 12).

TABLE 12
Different OTR and WVTR values for different types of paper (A and C)
which are modified with a BC suspension coating and optionally with
an additional layer of silicon oxide or nitrocellulose.

| | | | Dry | OTR | WVTR |
|---|---|---|---|---|---|
| | | | Coating | ranges, | ranges |
| | | | thickness, | ml/m² · | g/m² · day |
| Paper | Formula | Added layer | μm | day · atm | 50% RH |
| A, | Suspension | — | 40 | 14.4-47 | 147-154.6 |
| MONDI | D/E | | | | |
| 60 g/m² | | | | | |
| A, | Suspension | Silicon oxide | 40 | 11.2-24.2 | 90.8-94.6 |
| MONDI | D/E | | | | |
| 60 g/m² | | | | | |
| C, | Suspension | — | 40 | 2.8-7.1 | 147.6-154.6 |
| NATURA | D/E | | | | |
| 120 g/m² | | | | | |
| C, | Suspension | Nitro- | 40 | 3.2-4.6 | 90.8-94.6 |
| NATURA | D/E | cellulose | | | |
| 120 g/m² | | | | | |

As seen from table 12:
There is no noticeable difference in performance whether BC suspension D or E is used, thus the BC solids concentration range 1.6-2.3% performed the same as coating.
The use of silicon oxide as a second layer did not have an incremental effect on the OTR of the coated MONDI paper, when compared to the use of a single layer of BC suspension.
Coating with nitrocellulose as second layer decreased the NATURA paper's OTR by 50% compared to the use of a single layer of BC suspension.
The use of silicon oxide deposition or nitrocellulose as second layer (added layer) decrease the WVTR 50% compared to the value for the paper without the second layer.

### Example 6—Application of One-Phase BC/SiO₂ Suspension

The use of two coating layers to achieve a certain coating performance can be troublesome as it increases time and resources for applying the coating. Application of a single layer coating is more preferable. Thus, we formulated one phase water (or other polar solvent) BC-based suspensions which combined the two layers (BC with nitrocellulose or silicon dioxide). In this example we used food-grade silicon dioxide particles as additives. Nitrocellulose was not used further as it cannot be dissolved in water which is the preferred solvent for paper (or any other fibre-based material) coatings. Furthermore, the use of ether or other organic solvents is dangerous in higher scale if not handled properly.

### Objective

To use water-based one-phase BC/SiO₂ suspensions for coating. Both different modified and unmodified SiO₂ was tested.

### Material and Methods and Results

We used two types of papers for coating; paper A (MONDI, 60 g/m²) and paper D (Fedrigoni, 360 g/m²).

Three references of silicon dioxide (SiO₂) particles were selected. Two silicon dioxide particles modified with hydrophobic character and one unmodified hydrophilic silicon dioxide particle as a blank:
Modified SiO₂ with dimethylsylilates H15 particles with BET surface of 130-170 m²/g (SiO₂ H15).
Modified SiO₂ with dimethylsylilates H18 particles with BET surface of 170-230 m²/g (SiO₂ H18).
Unmodified SiO₂ particles with a specific surface area of 200 m²/g.

All the selected silicon dioxide particles were food grade. The silicon dioxide particles were produced via flame hydrolysis prior to modification with dimethylsylilates H15 and H18 (food-grade, Wacker). Both modified silicon dioxide particles are white colloidal powders with a density of 2.2 g/cm³. On the other hand, the unmodified SiO₂ particles (food-grade AEROSIL 200, Wacker) are hydrophilic fumed silicas.

Preparation of water-based one-phase BC/SiO₂ suspensions:
Unmodified or modified SiO₂ particles (described above) were dispersed in a 90% ethanol solution to obtain a 2-4% wt SiO₂ solid content before the addition to BC suspensions.
After 24 hours of reaction, the BC/SiO₂ solutions were filtered to remove excess chemicals and silicon dioxide particles. Finally, the total solid content of the filtered BC/SiO₂ solutions were determined by the oven drying method to be around 10-15%.
The BC/SiO₂ solutions were evaluated in terms of chemical structure (FTIR) and thermal stability (TGA) to confirm the success of the chemical modifications.
   a. Application of One-Phase BC/SiO₂ Suspension to Paper A

One phase BC/SiO₂ suspension were applied onto the paper substrate (paper A) at laboratory scale using an automatic Film Applicator (Standard AB4400) with perforated heated vacuum bed. Before application, the substrate (paper) was held securely by clamps and also vacuum was used, then the coating formulation was spread as a consistent and reproducible film across the surface with the aid of an applicator. Based on results obtained in previous experiments, the wet coating thickness was evaluated to be 100 μm.

Grammage, roughness (or smoothness), porosity, water absorption were determined as previously described herein.

### FTIR for Modification Evaluation

The sample is prepared by a random part of the sample (to ensure the representativeness of the sample). After that, the sample is introduced into a diamond-tipped screw and the infrared spectrum is determined. The tests are performed in duplicate. ATR FT-IR spectroscopy was conducted over the wavenumber range 650-4000 cm⁻¹. 16 scans were taken for each spectrum at a resolution of 4 cm⁻¹. Modified SiO₂ will show peak at 810 and 1180 cm⁻¹. Successful modification of BC with SiO₂ will result a symmetric vibration of Si—O—Si and Si—C link at 1570 cm⁻¹.

### Thermogravimetric Analysis (TGA) for Modification Evaluation

TGA measures the mass variation of a sample when undergoing temperature changes for a certain time and under a controlled atmosphere. These measurements are mainly used to determine the composition of materials and predict their thermal stability at temperatures up to 1000° C. This technique can characterize materials that show both loss and weight gain such as due to either decomposition, oxidation and/or dehydration.

Process parameters for TGA:
Sample: 3-6 mg.
Initial Atmosphere: Nitrogen.
Initial Temperature: 25° C.
Ramp from 20° C./min to 900° C.
Atmosphere change: Oxygen
Isothermal at 900° C. for 10 minutes

### Water Contact Angle

When an interface exists between a liquid and a solid, the angle between the surface of the liquid and the outline of the contact surface is described as the contact angle 6 (lower case theta). The contact angle (wetting angle) is a measure of the wettability of a solid by a liquid. In the case of complete wetting the contact angle is 0°. Between 0° and 90°, the solid is wettable and if the contact angle is above 90° it is not wettable. In the case of ultrahydrophobic materials with the so-called lotus effect, the contact angle approaches the theoretical limit of 180°.

The wetting angle is determined following the standard ASTM D724-99 and using a contact angle goniometer as equipment.

TABLE 13
Evaluation of one phase BC suspension
D/modified SiO₂ using FTIR and TGA

| | | TGA, |
|---|---|---|
| Samples | FTIR | Tmax (° C.) |
| SiO₂ H15 or H18 | Peak at 810 and 1180 cm⁻¹ | 353 |
| BC with SiO₂ H18 | Peak at 1570 cm⁻¹ | 320-340 |
| BC with SiO₂ H15 | Peak at 1570 cm⁻¹ | 340-350 |

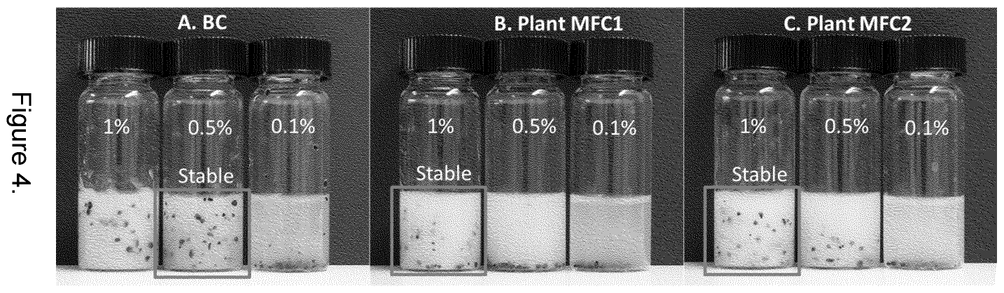

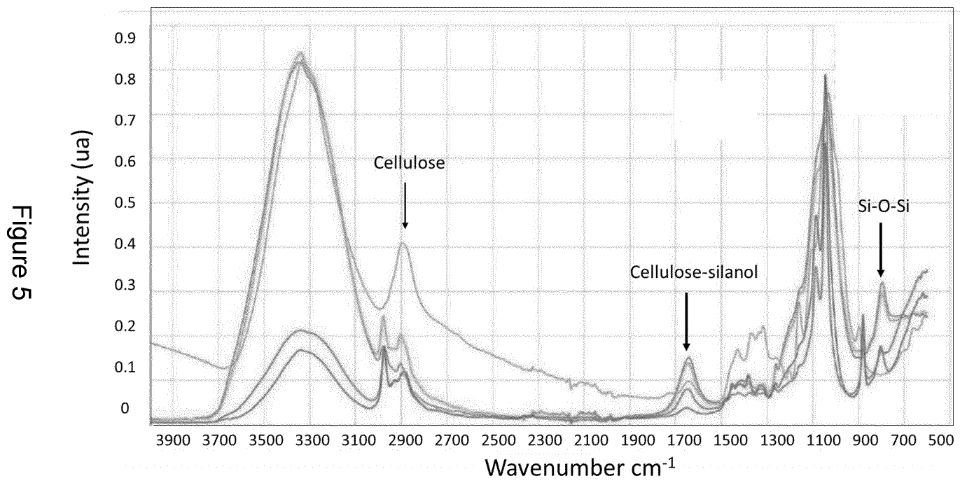

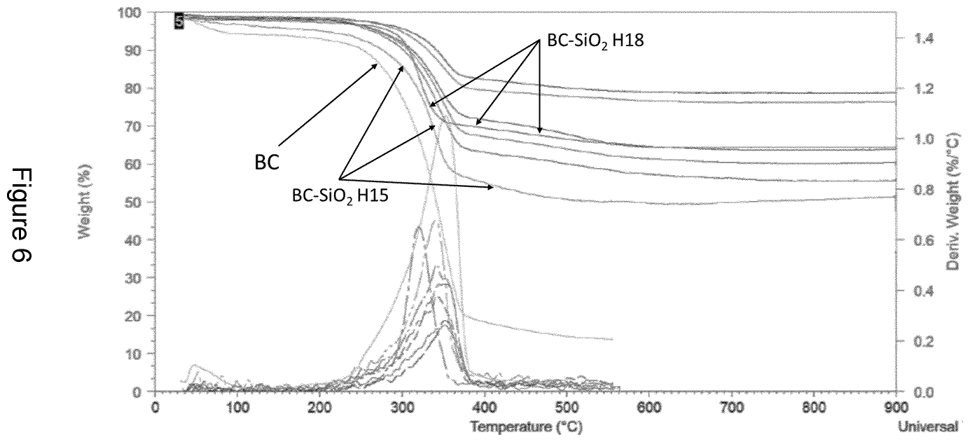

From Table 13, it can be concluded that:
Where the silane modification is most clearly evidenced is with the appearance of a FTIR band around 1570 cm⁻¹, corresponding to the deformation vibrations of the cellulose groups linked to silanol, and with the appearance of the bands around 810 cm⁻¹ associated with the symmetric vibration of the Si—O—Si and asymmetric links of the Si—C link (FIG. 5).
As can be observed, the thermal stability (TGA) of the BC is almost the same with the different modification processes evaluated (FIG. 6).

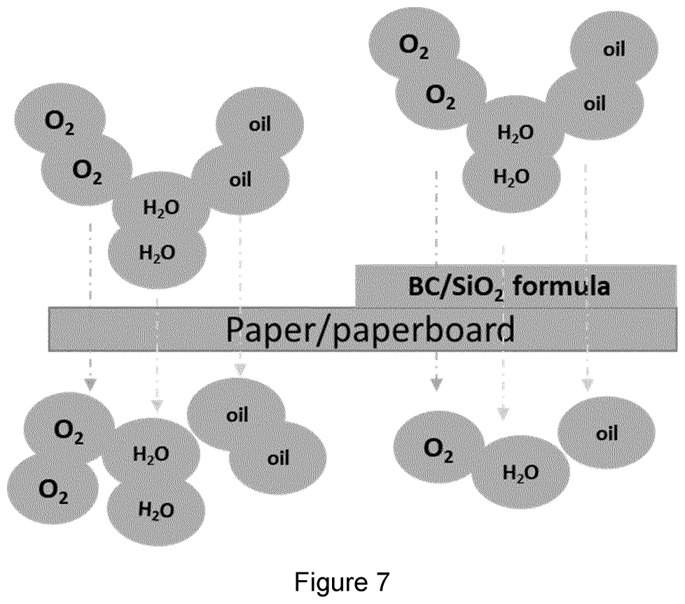

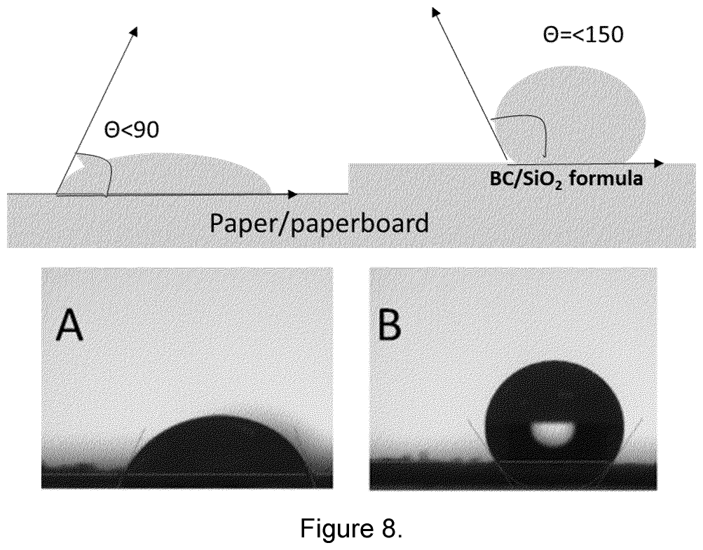

The effect of water-based one phase BC suspension D/un- or modified SiO₂ coating to paper A's grammage, roughness, porosity, water absorption and water contact angle were evaluated (FIG. 7, FIG. 8 and Table 14).

TABLE 14
Effect of water-based one phase BC suspension D/un- or modified SiO₂ coating
to paper A's grammage, roughness, porosity, water absorption and water contact
angle. The wet coating thickness was 100 μm for all suspension formulas,
except Paper A (no suspension formula, first row) where no coating were applied.

| | BC solid | | | | Water | Water |
|---|---|---|---|---|---|---|
| Suspension | content, | Grammage | Roughness | Porosity | absorption | contact |
| formula | % | (g/m²) | (mL/min) | (mL/min) | (g/m²) | angle, ° |
| Paper A | — | 58.8 ± 0.1 | 630 ± 2.3 | 283 ± 2.1 | 23.2 ± 2.3 | 88.6 ± 10.3 |
| Paper A Susp. D | 2 | 60.7 ± 0.1 | 537 ± 2.3 | 108 ± 4.3 | 70.8 ± 1.2 | 11.6 ± 4.2 |
| Unmodified SiO₂ | | | | | | |
| Paper A Susp. D | 2 | 62.5 ± 0.1 | 473 ± 3.3 | 93 ± 2.5 | 25.3 ± 1.5 | 72.3 ± 14.3 |
| SiO₂ H15 | | | | | | |
| Paper A Susp. D | 2 | 60.5 ± 0.1 | 446 ± 3.3 | 193 ± 3.4 | 15.4 ± 4.3 | 113.8 ± 11.2 |
| SiO₂ H18 1:2 | | | | | | |
| Paper A Susp. D | 4 | 63.6 ± 0.1 | 221.2 ± 4.3 | 178 ± 2.5 | 7.8 ± 4.3 | 129.7 ± 21.2 |
| SiO₂ H18 1:2 | | | | | | |
| Paper A Susp. D | 4 | 64.4 ± 0.1 | 344.5 ± 3.3 | 117 ± 3.3 | 18.6 ± 6.3 | 122 ± 11.3 |
| SiO₂ H18 1:1 | | | | | | |
| Paper A Susp. D in | 2 | 60.5 ± 0.1 | 446 ± 2.1 | 90 ± 5.5 | 22-25 ± 3.4 | 140 ± 13.7 |
| ethanol SiO₂ H18 | | | | | | |
| 1:1 (H-BC) | | | | | | |
| Paper A Susp. D in | 2 | 60.5 ± 0.1 | 446 ± 1.3 | 77 ± 2.3 | 17-20 ± 4.1 | 140 ± 5.6 |
| ethanol SiO₂ H18 | | | | | | |
| 1:1 + 1% PVOH | | | | | | |
| (H-BC/PVOH1%) | | | | | | |

From Table 14 we can conclude that:
Coating with water-based one phase BC suspension D/un- or modified SiO₂ suspensions of paper A decreases the surface roughness of the paper in general.
The use of unmodified SiO₂ in the BC suspension did not decrease the water absorption of paper A and affected the water contact angle negatively.
The use of SiO₂ H18 increases the water contact angle and decreases the water absorption of coated paper A's surface.
H-BC (BC suspension D in ethanol SiO₂ H18 1:1) with 2% solid content is the best coating formula as it decreases porosity the most. The addition of 1% PVOH to H-BC decreases the variability of the coating performance, evaluated by variability of the water contact angle. PVOH increases the elasticity of the BC-SiO₂ based suspension, thus preventing microcracking.
Modified SiO₂ with longer sylilate tails, such as SiO₂ H20 and SiO₂ H30, have also been tested, but for those a worse performance than for SiO₂ H15 was found, when the barrier performance on coated paper A was evaluated based on the measurement of the water contact angle.
   b. Application of One-Phase BC/Modified SiO₂ Suspension to Paper D

Sample H-BC (BC suspension D in ethanol SiO₂ H18 1:1) and prepared coatings H-BC+1% PVOH, H-BC+3% PVOH, H-BC+5% PVOH were applied on basic board 360 g/m² (Paper D) with film press coating application usind Sumet coater. The process parameters were optimized and is presented in Table 15.

TABLE 15
Process parameters for application of
samples/coatings on paper D surfaces

| | H-BC with or |
|---|---|
| Samples | without PVOH |
| Pressure on rod (N) | 15 |
| Pressure on roll (N) | 50 |
| Speed (m/min) | 7 |
| drying | IR: 100%, |
| | hot air: 140 C. |
| Time of drying | 1.5 minutes |

The effect of water-based one phase BC suspension D/modified SiO₂ suspension coating to paper D's grammage, grease resistance, oil absorption, water absorption, WVTR at 50 and 85% RH is shown in Table 16.1 and Table 16.2. Table 16.1 and Table 16.2 belong together and Table 16.2 is a continuation of Table 16.1.

TABLE 16.1
Effect of water-based one phase BC suspension
D/modified SiO₂ suspension coating to paper D’s grammage.

| | | | Basic | Coated | Coat |
|---|---|---|---|---|---|
| Suspension | Viscosity, | pH | board | board | weight |
| formula | mPas | value | g/m² | g/m² | g/m² |
| Basic paper | — | — | 365.7 | — | 0 |
| Paper/H-BC | 116 | 9.37 | 365.7 | 369.8 | 4.1 |
| Paper/H- | 413 | 7.35 | 365.7 | 364 | 3.9 |
| BC/PVOH1% | | | | | |
| Paper/H- | 428 | 7.68 | 365.7 | 362.7 | 4.1 |
| BC/PVOH3% | | | | | |
| Paper/H- | 465 | 8.11 | 365.7 | 358.2 | 3.7 |
| BC/PVOH5% | | | | | |

TABLE 16.2
Effect of water-based one phase BC suspension D/modified SiO₂ suspension
coating to paper D’s grease resistance, oil absorption, water absorption,
WVTR at 50 and 85% RH. Table 16.2 is a continuation of Table 16.1.

| | | Oil | | | |
|---|---|---|---|---|---|
| | | absorption | Water | WVTR | WVTR |
| | Grease | Cobb | absorption | g/m² · day | g/m² · day |
| Suspension | resistance | Unger, | Cobb, | at 85% | at 50% |
| formula | KIT | g/m² | g/m² | RH | RH |
| Basic paper | 4 | 83.5 | 26 | 820 | 400 |
| Paper/H-BC | 6 | 40.4 | 24.6 | 570 | 40 |
| Paper/H- | 9 | 8.5 | 26.6 | 240 | 44 |
| BC/PVOH1% | | | | | |
| Paper/H- | 8 | 10.4 | 27.9 | 400 | 50 |
| BC/PVOH3% | | | | | |
| Paper/H- | 8 | 27.2 | 25.7 | 440 | 55 |
| BC/PVOH5% | | | | | |

From Table 16.1 and Table 16.2 it can be concluded that:
Increased percentage addition of PVOH to H-BC suspension increases the viscosity of the suspension.
The highest grease resistance was achieved with H-BC with 1% PVOH due to its hydrophilic nature, which is also decreasing oil absorption.
Addition of 1% PVOH decreases WVTR, both at 50% and 85% RH as PVOH introduces elasticity to rigid structures of H-BC where micro-scale cracks can be easily induced.

### REFERENCES

Martien van den Oever, Karin Molenveld, Maarten van der Zee, Harriette Bos. Bio-based and biodegradable plastics—Facts and Figures.
Choi & Shin. (2020) The Nanofication and Functionalization of Bacterial Cellulose and Its Applications. Nanomaterials. 10(3):406. doi:10.3390/nano10030406.
Diofan® tecnical sheet. https://www.solvay.com/en/product/diofan-736
Eval® technical sheet. https://eval.kuraray.com/information-center/datasheets/
Fapyane and Ferapontova (2017). Electrochemical Assay for a Total Cellulase Activity with Improved Sensitivity. Anal. Chem. 2017, 89, 7, 3959-3965
O'Connor et al. (1958) Applications of infrared absorption spectroscopy to investigations of cotton and modified cottons: Part I: physical and crystalline modifications and oxidation. Text Res J. 28(5):382-392.
Gray. (2018) What is the real price of getting rid of plastic packaging? BBC. https://www.bbc.com/worklife/article/20180705-whats-the-real-price-of-getting-rid-of-plastic-packaging.
Queirós Dourado & Portela de Gama. (2020) Bacterial cellulose formulations, methods and uses thereof. WO 2020/136629. Filing date 30 Dec. 2020.
Raghavendran, Vijayendran et al. (2020) Chapter Three—Bacterial cellulose: Biosynthesis, production, and applications. Advances in microbial physiology. Volume 77: p 89-138.
Tadahiko et al. (1998) Bacterial cellulose concentrate and method for treating said concentrate. EP0841345. Filing date 24 May 1996; Issue date 15 Jan. 2003.
Vaisanen et al. (2018) Quantification of accessible hydroxyl groups in cellulosic pulps by dynamic vapor sorption with deuterium exchange. Cellulose. 25, 6923-6934.
Watanabe et al. (1997) Method for processing bacterial cellulose. U.S. Pat. No. 6,153,413. Filing date 21 Jun. 1996; Issue date 28 Nov. 2000.
World Wildlife Fund (2019). Solving plastic pollution through accountability. ISBN 978-2-940529-93-3.

### Items

1. A bacterial cellulose (BC) suspension comprising 0.5 to 4% (w/v) of BC solids, such as between 0.5 and 1% (w/v) of BC solids, such as between 1 and 2% (w/v) of BC solids, such as between 2 and 3% (w/v) of BC solids, such as between 3 and 4% (w/v) of BC solids.
2. A bacterial cellulose (BC) suspension comprising 1.6 to 2.3% (w/v) BC solids.
3. A bacterial cellulose (BC) suspension comprising 1.6 to 2.3% (w/v) BC solids, said suspension having a viscosity between 1 and 500 Pa·s at a shear stress of 1/s.
4. The BC suspension according to any one of the preceding items comprising between 1.6 and 2.2% (w/v) of BC solids, such as between 1.6 and 2.1% (w/v) of BC solids, such as between 1.6 and 2.0% (w/v) of BC solids, such as between 1.6 and 1.9% (w/v) of BC solids, such as between 1.6 and 1.8% (w/v) of BC solids, such as between 1.6 and 1.7% (w/v) of BC solids, such as between 1.7 and 2.3% (w/v) of BC solids, such as 1.7 and 2.2% (w/v) of BC solids, such as between 1.7 and 2.1% (w/v) of BC solids, such as between 1.7 and 2.0% (w/v) of BC solids, such as between 1.7 and 1.9% (w/v) of BC solids, such as between 1.7 and 1.8% (w/v) of BC solids, such as between 1.8 and 2.3% (w/v) of BC solids, such as between 1.8 and 2.2% (w/v) of BC solids, such as between 1.8 and 2.1% (w/v) of BC solids, such as between 1.8 and 2.0% (w/v) of BC solids, such as between 1.8 and 1.9% (w/v) of BC solids, such as between 1.9 and 2.3% (w/v) of BC solids, such as between 1.9 and 2.2% (w/v) of BC solids, such as between 1.9 and 2.1% (w/v) of BC solids, such as between 1.9 and 2.0% (w/v) of BC solids, such as between 1.6 and 1.9% (w/v) or between 2.1 and 2.3% (w/v).
5. The BC suspension according to any one of the preceding items, wherein the BC solids in said BC suspension are dispersed in fiber clusters.
6. A bacterial cellulose (BC) suspension comprising 1.6 to 2.3% (w/v) BC solids, said suspension comprising BC fiber clusters having a size between 10 nm and 1500 μm.
7. A bacterial cellulose (BC) suspension comprising 1.6 to 2.3% (w/v) BC solids, and/or said suspension comprising BC fiber clusters having a size between 10 nm and 1500 μm, and/or said suspension having a viscosity between 1 and 500 Pa·s at a shear stress of 1/s.
8. A bacterial cellulose (BC) suspension comprising 1.6 to 2.3% (w/v) BC solids, said suspension comprising BC fiber clusters having a size between 10 nm and 1500 μm, said suspension having a viscosity between 1 and 500 Pa·s at a shear stress of 1/s.
9. The BC suspension according to any one of the preceding items, wherein the size of the BC fiber clusters varies between 10 nm to 1500 μm, such as 10 nm to 1000 μm, such as 10 nm to 800 μm, such as 50 nm to 500 μm, such as 50 nm to 400 μm, such as 50 nm to 100 μm, such as 50 nm to 10 μm, such as 50 to 1000 nm, such as 50 to 100 nm, such as 10 to 100 μm, such as 10 nm to 100 μm.
10. The BC suspension according to any one of the preceding items, wherein the viscosity of said BC suspension is 2 to 500 Pa·s at a shear stress of 1/s, such as between 2 and 500 Pa·s, such as between 2 and 99 Pa·s, such as between 100 and 199 Pa·s, such as between 200 and 299 Pa·s, such as between 300 and 399 Pa·s, such as between 400 and 500 Pa·s.
11. The BC suspension according to any one of the preceding items, comprising BC suspended in a protic solvent, such as water.
12. The BC suspension according to any one of the preceding items, wherein at least some of the accessible chemical groups of the BC are functionalized, such as 1 to 80%, such as 5 to 80%, such as 10 to 80%, such as 20 to 80%, such as 30 to 80%, such as 40 to 80%, such as 50 to 80%, such as 60 to 80%; such as 70 to 80%, preferably wherein the accessible chemical groups are accessible hydroxyl groups.
13. The BC suspension according to any one of the preceding items, wherein at least some of the accessible hydroxyl groups of the BC are substituted with nitro groups.
14. The BC suspension according to any one of the preceding items, wherein at least some of the accessible hydroxyl groups of the BC are acetylated.
15. The BC suspension according to any one of the preceding items, wherein at least some of the accessible hydroxyl groups of the BC are substituted with nitro groups, optionally, wherein 1 to 80% of the accessible hydroxyl groups are substituted with nitro groups, such as 5 to 80%, such as 10 to 80%, such as 20 to 80%, such as 30 to 80%, such as 40 to 80%, such as 50 to 80%, such as 60 to 80%, such as 70 to 80%.
16. The BC suspension according to any one of the preceding items, wherein at least some of the accessible hydroxyl groups of the BC are acetylated, optionally, wherein 1 to 80% of the accessible hydroxyl groups are acetylated, such as 5 to 80%, such as 10 to 80%, such as 20 to 80%, such as 30 to 80%, such as 40 to 80%, such as 50 to 80%, such as 60 to 80%, such as 70 to 80%.
17. The BC suspension according to any one of the preceding items, wherein 1 to 80% of the accessible hydroxyl groups are substituted with nitro groups, such as 5 to 80%, such as 10 to 80%, such as 20 to 80%, such as 30 to 80%, such as 40 to 80%, such as 50 to 80%, such as 60 to 80%; such as 70 to 80%.
18. The BC suspension according to any one of the preceding items, wherein 1 to 80% of the accessible hydroxyl groups are acetylated, such as 5 to 80%, such as 10 to 80%, such as 20 to 80%, such as 30 to 80%, such as 40 to 80%, such as 50 to 80%, such as 60 to 80%; such as 70 to 80%.
19. The BC suspension according to any one of the preceding items, further comprising an additional component, such as glycerol, polyvinyl alcohol (PVOH), pectin, casein, starch, silicon oxide, and/or titanium oxide.
20. The BC suspension according to any one of the preceding items, further comprising an additional component, such as unmodified or modified silicon oxide (SiO).
21. A method for producing a BC suspension comprising the steps of:
   a) Incubating at least one cellulose producing bacteria in a culture medium, and optionally, at least one other microorganism which does not produce cellulose, thereby obtaining BC in said culture medium;
   b) Recovering said BC;
   c) Optionally, contacting said BC with a functionalization agent, wherein said functionalization agent is selected from nitric acid and acetic anhydride, thereby functionalizing said BC;
   d) Converting the recovered and/or the functionalized BC into a BC suspension comprising 0.5 to 4.0% (w/v) BC solids, such as between 0.5 and 1% (w/v) of BC solids, such as between 1 and 2% (w/v) of BC solids, such as between 2 and 3% (w/v) of BC solids, such as between 3 and 4% (w/v) of BC solids, preferably between 1.6 and 2.3% (w/v) BC solids.
22. The method according to item 21, wherein the at least one cellulose producing bacteria is Komagataeibacter rhaeticus and/or Gluconobacter oxydans.
23. The method according to any one of items 21 to 22 wherein the functionalization increases the hydrophobicity of the BC, wherein the hydrophobicity is defined as the water contact angle of the BC.
24. The method according to any one of items 21 to 23, wherein the water contact angle of the BC is increased from 450 to 150°, such as to 50°, such as to 60°, such as to 70°, such as to 80°, such as to 90°, such as to 100°, such as to 110°, such as to 120°, such as to 130°, such as to 140°.
25. The method according to any one of items 21 to 24, wherein the BC suspension is prepared by comminution of the recovered and/or the functionalized BC.
26. The method according to any one of items 21 to 25, wherein the culture medium comprises a carbon source, selected from glycerol, fructose, sucrose and/or glucose.
27. The method according to any one of items 21 to 26, wherein the culture medium comprises juice from vegetables and/or fruits, such as juice from apple, strawberry, blackberry, aronia, chokeberry, grape, plum, cranberry, pea, orange, banana, potato, carrot, sugar beet and/or combinations thereof.
28. The method according to any one of items 21 to 27, wherein the culture medium comprises biomass waste, such as molasses and/or plant waste.
29. The method according to any one of items 21 to 28, wherein the culture medium comprises sucrose at a concentration of 1 to 30%, such as 2 to 10%, such as 2 to 5%, such as 1 to 8%; or such as 4 to 10%, or such as 8 to 15%, or such as 10 to 25%, or such as 15 to 20%.
30. The method according to any one of items 21 to 29, wherein the culture medium comprises glucose at a concentration of 1 to 30%, such as 2 to 10%, such as 2 to 5%, such as 1 to 8%; or such as 4 to 10%, or such as 8 to 15%, or such as 10 to 25%, or such as 15 to 20%.
31. The method according to any one of items 21 to 30, wherein the culture medium comprises one or more antioxidants and/or polyphenols extracted from fruits, vegetables, and/or tea, such as black tea.
32. The method according to any one of items 21 to 31, wherein the culture medium comprises theaflavins and/or derivatives thereof, preferably wherein the derivative comprises a tropolone moiety.
33. The method according to any one of items 21 to 32, wherein the culture medium comprises theaflavins selected from the group consisting of theaflavin, theaflavin-3-gallate, theaflavin-3′-gallate, and/or theaflavin-3-3′-digallate.
34. The method according to any one of items 21 to 33, wherein the culture medium comprises one or more polyphenols, such as theaflavins, at a concentration between 5 and 100 g/L, such as between 5 and 90 g/L, such as between 10 and 80 g/L, such as between 15 and 75 g/L, such as between 20 and 70 g/L, such as between 25 and 65 g/L, such as between 30 and 60 g/L, such as between 35 and 55 g/L, such as between 40 and 50 g/L, such as about 45 g/L.
35. The method according to any one of items 21 to 34, wherein the culture medium is not sterilized prior to step a).
36. The method according to any one of items 21 to 35, wherein the culture medium has a pH value between 2.5 and 4.0, such as between 2.6 and 3.9, such as between 2.7 and 3.8, such as between 2.8 and 3.7, such as between 2.9 and 3.6, such as between 3.0 and 3.5, such as between 3.1 and 3.4, such as between 3.2 and 3.3.
37. The method according to any one of items 21 to 36, wherein the at least one cellulose producing bacteria, and optionally the at least one other microorganism which does not produce cellulose, is isolated from or derived from a Kombucha tea biofilm.
38. The method according to any one of items 21 to 37, wherein the at least one cellulose producing bacteria is Komagataeibacter rhaeticus and/or Gluconobacter oxydans, and optionally wherein the at least one microorganism which does not produce cellulose is Hanseniaspora uvarum, Brettanomyces bruxellensis, Zygosaccharomyces bailii and/or Kloeckera lindneri.
39. The method according to any one of items 21 to 38, wherein step a) is performed at a temperature between 15 and 30° C., such as between 18 and 28° C., such as between 20 and 26° C., such as between 22 and 24° C.
40. The method according to any one of items 21 to 39, wherein step a) is performed at room temperature.
41. The method according to any one of items 21 to 40, wherein the cultivation in step a) is an aerobic fermentation, such as a static aerobic fermentation, such as without shaking.
42. The method according to any one of items 21 to 42, wherein the cultivation in step a) is an agitated fermentation.
43. The method according to any one of items 21 to 41, wherein the cultivation in step a) is performed for a period of 2 to 10 days, such as between 2 and 9 days, such as between 3 and 8 days, such as between 4 and 7 days, such as between 5 and 6 days.
44. The method according to any one of items 21 to 43, wherein the BC produced in step a) is in the form of a BC pellicle.
45. The method according to item 44, further comprising a step of recovering the BC, preferably by removing it from the surface of the culturing medium or from the interface between surface and culturing medium.
46. The method according to any one of items 44 to 45, wherein the BC pellicle has a thickness between 0.2 and 2 cm, such as between 0.2 and 1.8 cm, such as between 0.4 and 1.6 cm, such as between 0.6 and 1.4 cm, such as between 0.8 and 1.2 cm, such as about 1.0 cm.
47. The method according to any one of items 21 to 46, wherein the BC has a crystallinity index of 0.05 to 3, such as between 0.1 and 2.9, such as between 0.2 and 2.8, such as between 0.3 and 2.7, such as between 0.4 and 2.6, such as between 0.5 and 2.5, such as between 0.6 and 2.4, such as between 0.6 and 2.3, such as between 0.7 and 2.2, such as between 0.8 and 2.1, such as between 0.9 and 2.0, such as between 1.0 and 1.9, such as between 1.1 and 1.8, such as between 1.2 and 1.7, such as between 1.3 and 1.6, such as between 1.4 and 1.5.
48. The method according to any one of items 21 to 47, wherein the BC has a degree of polymerization (DP) of 900 to 2500, such as between 1000 and 2400, such as between 1200 and 2200, such as between 1400 and 2000, such as between 1600 and 1800.
49. The method according to any one of items 21 to 48, further comprising a step of sterilizing the BC.
50. The method according to any one of items 21 to 49, further comprising the step of contacting the BC with a base, such as NaOH and/or NaClO₄, thereby sterilizing the BC, preferably after recovering the BC.
51. The method according to any one of items 21 to 50, further comprising the step of contacting the BC with a base, preferably after recovering the BC, such as 0.3 to 10% (w/w) NaOH, such as with 0.5 to 9%, such as 0.75 to 8%, such as 1 to 7%, such as 2 to 6%, such as 3 to 5% NaOH., thereby sterilizing the BC.
52. The method according to any one of items 10 to 51, comprising step c) wherein the functionalization agent is nitric acid, whereby at least some of the accessible hydroxyl groups of the BC are substituted with nitro groups, preferably wherein the concentration of nitric acid is 0.3 to 10% (v/v), such as 1 to 10%, such as 1 to 5%, such as 2 to 6%, such as 3 to 8%.
53. The method according to any one of items 10 to 52, comprising step c) wherein the functionalization agent is acetic anhydride, whereby at least some of the accessible hydroxyl groups of the BC are acetylated, preferably wherein the concentration of acetic anhydride is 1 to 25%, such as 5 to 15%, such as 1 to 10%, such as 20 to 25%.
54. The method according to any one of items 25 to 53, wherein the recovered and/or the functionalized BC is comminuted using at least one of the comminution methods and/or instruments selected from the group consisting of mechanical shearing stress, wet grinding mill, cutting, crushing, trituration, ultrasonication, aqueous counter-collision, high-pressure homogenization, microfluidizer, comminution using an IKA Turrax, and comminution using a supermasscolloider, such as Masuko grinding stone.
55. The method according to any one of items 25 to 54, wherein the comminution comprises at least two of the comminution methods and/or instruments selected from the group consisting of mechanical shearing stress, wet grinding mill, cutting, crushing, trituration, ultrasonication, aqueous counter-collision, high-pressure homogenization, comminution using an IKA Turrax, and comminution using a supermasscolloider, such as Masuko grinding stone.
56. The method according to any one of items 25 to 55, wherein the comminution comprises grinding the recovered BC, such as grinding with a supermasscolloider, such as a Masuko grinding stone, at a speed of 500 to 3000 rpm, between 600 and 2800 rpm, such as between 700 and 2600 rpm, such as between 800 and 2400 rpm, such as between 900 and 2200 rpm, such as between 1000 and 2000 rpm, such as between 1200 and 1800 rpm, such as between 1400 and 1600 rpm.
57. The method according to any one of items 25 to 56, further comprising a step of centrifugating the BC before and/or after the comminution.
58. The method according to any one of items 25 to 57, further comprising a step of heating the BC after and/or during the comminution.
59. The method according to item 58, wherein the heating is performed at temperature between 30° C. and 200° C., such as between 30° C. and 200° C., such as between 40° C. and 190° C., such as between 50° C. and 180° C., such as between 60° C. and 170° C., such as between 70° C. and 160° C., such as between 80° C. and 150° C., such as between 90° C. and 140° C., such as between 100° C. and 130° C., such as between 110° C. and 120° C.
60. The method according to any one of items 58 and 59, wherein the heating is performed for 5 min to 10 h, such as 10 min to 9 h, such as 20 min to 8 h, such as 30 min to 7 h, such as 1 h to 6 h, such as 2 h to 5 h, such as 3 h to 4 h.
61. The method according to any one of items 25 to 60, wherein the comminution generates a BC suspension as defined in any one of items 1 to 19.
62. A BC suspension obtained from the method according to any one of items 21 to 61.
63. The BC suspension according to item 62, wherein said BC suspension is as defined in any one of items 1 to 19.
64. Use of the BC suspension as defined in any one of the preceding items for coating a material, such as the surface of a material.
65. The use according to item 64, wherein the material is selected from the group of materials consisting of paper, cardboard, foodstuff, cosmetics and textiles, such as woven and/or non to woven textiles.
66. The use according to any one of items 64 to 65, wherein the coating thickness is between 30 and 110 μm, such as between 30 and 80 μm, such as between 50 and 90 μm, such as between 60 and 100 μm, such as between 40 and 70 μm.
67. The use according to any one of items 64 to 66, wherein coating paper or cardboard with the BC suspension generates a coated paper or cardboard with a grammage between 45 to 70 g/m², such as 45 and 50 g/m², or between 50 and 55 g/m², or between 55 and 60 g/m², or between 60 and 65 g/m², or between 65 and 70 g/m².
68. The use according to any one of items 64 to 67, wherein coating paper or cardboard with the BC suspension generates a coated paper or cardboard with a smoothness between 200 to 1000 mL/min, such between 200 and 500 mL/min, between 300 and 600 mL/min, between 400 and 700 mL/min, between 500 and 1000 mL/min, between 600 and 900 mL/min, or between 400 and 800 mL/min.
69. The use according to any one of items 64 to 68, wherein coating paper or cardboard with the BC suspension generates a coated paper or cardboard with a porosity between 50 to 250 mL/min, such as such as between 65 and 105 mL/min, such as between 95 and 150 mL/min, such as between 75 and 175 mL/min, such as between 100 and 200 mL/min, such as between 50 and 145 mL/min.
70. The use according to any one of items 64 to 69, wherein coating paper or cardboard with the BC suspension generates a coated paper or cardboard with a water absorption of 15 to 25 g/m², such as between 16 and 24 g/m², such as between 17 and 23 g/m², such as between 18 and 22 g/m², such as between 19 and 21 g/m².
71. The use according to any one of items 64 to 70, wherein coating the material with the BC suspension generates a coated paper or cardboard with an oxygen transmission rate (OTR) between 1 to 60 ml/m²·day·atm, such as between 2 and 7 ml/m²·day·atm, such as between 15 and 50 ml/m²·day·atm, such as between 10 and 25 ml/m²·day·atm, such as 3 and 5 ml/m²·day·atm.
72. The use according to any one of items 64 to 71, wherein the material is coated with a BC suspension after being coated with another agent, optionally, wherein the material is coated with another agent after being coated with a BC suspension.
73. The use according to item 72, wherein the other agent is polyvinyl alcohol (PVOH), nitrocellulose and/or silicon oxide.

1. A bacterial cellulose (BC) suspension comprising 1.6 to 2.3% (w/v) BC solids, said suspension comprising BC fiber clusters having a size between 10 nm and 1500 μm, said suspension having a viscosity between 1 and 500 Pa·s at a shear stress of 1/s.

2. The BC suspension according to claim 1 comprising between 1.6 and 2.2% (w/v) of BC solids, such as between 1.6 and 2.1% (w/v) of BC solids, such as between 1.6 and 2% (w/v) of BC solids, such as between 1.6 and 1.9% (w/v) of BC solids, such as between 1.6 and 1.8% (w/v) of BC solids, such as between 1.6 and 1.7% (w/v) of BC solids, such as between 1.7 and 2.3% (w/v) of BC solids, such as 1.7 and 2.2% (w/v) of BC solids, such as between 1.7 and 2.1% (w/v) of BC solids, such as between 1.7 and 2.0% (w/v) of BC solids, such as between 1.7 and 1.9% (w/v) of BC solids, such as between 1.7 and 1.8% (w/v) of BC solids, such as between 1.8 and 2.3% (w/v) of BC solids, such as between 1.8 and 2.2% (w/v) of BC solids, such as between 1.8 and 2.1% (w/v) of BC solids, such as between 1.8 and 2.0% (w/v) of BC solids, such as between 1.8 and 1.9% (w/v) of BC solids, such as between 1.9 and 2.3% (w/v) of BC solids, such as between 1.9 and 2.2% (w/v) of BC solids, such as between 1.9 and 2.1% (w/v) of BC solids, such as between 1.9 and 2.0% (w/v) of BC solids, such as between 1.6 and 1.9% (w/v) or between 2.1 and 2.3% (w/v) BC solids.

3. The BC suspension according to any one of the preceding claims, wherein the sizes of the BC fiber clusters varies between 0.5 and 1500 μm, such as between 0.5 and 1300 μm, such as between 1 and 200 μm, such as between 5 and 1400 μm, such as between 5 and 1350 μm, such as between 5 and 1300 μm, such as between 50 and 1400 μm, such as between 50 and 1350 μm, such as between 75 and 1300 μm, such as between 75 and 1300 μm, such as between 70 and 1250 μm, such as between 5 and 1240 μm.

4. The BC suspension according to any one of the preceding claims, wherein the median size of the BC fiber clusters varies between 50 and 100 μm, such as between 50 and 75 μm, such as between 75 and 100 μm.

5. The BC suspension according to any one of the preceding claims, comprising BC suspended in a protic solvent, such as water.

6. The BC suspension according to any one of the preceding claims, further comprising an additional component, such as glycerol, polyvinyl alcohol (PVOH), pectin, casein, starch, modified and/or unmodified silicon oxide, and/or titanium oxide.

7. The BC suspension according to claim 5, wherein the modified silicon oxide is silicon dioxide (SiO₂) modified with dimethylsylilates particles having a sylilate tail length of H8 to H22, such as a sylilate tail length of H10 to H20, such as a sylilate tail length of H15 to H20, such as sylilate tail length of H15 to H18, such as a sylilate tail length of H15, such as a sylilate tail length of H16, such as a sylilate tail length of H17, such as a sylilate tail length of H18, optionally the modified SiO₂ is selected from SiO₂ modified with dimethylsylilates H15 particles with BET surface of 130-170 m²/g and SiO₂ modified with dimethylsylilates H18 particles with BET surface of 170-230 m²/g.

8. The BC suspension according to any one of claims 5 to 7, comprising up to 5% polyvinyl alcohol (PVOH).

9. The BC suspension according to any one of the preceding claims, wherein at least some of the accessible chemical groups of the BC are functionalized, such as 1 to 80%, such as 5 to 80%, such as 10 to 80%, such as 20 to 80%, such as 30 to 80%, such as 40 to 80%, such as 50 to 80%, such as 60 to 80%; such as 70 to 80%, preferably wherein the accessible chemical groups are accessible hydroxyl groups.

10. The BC suspension according to any one of the preceding claims, wherein at least some of the accessible hydroxyl groups of the BC are substituted with nitro groups, optionally, wherein 1 to 80% of the accessible hydroxyl groups are substituted with nitro groups, such as 5 to 80%, such as 10 to 80%, such as 20 to 80%, such as 30 to 80%, such as 40 to 80%, such as 50 to 80%, such as 60 to 80%, such as 70 to 80%.

11. The BC suspension according to any one of the preceding claims, wherein at least some of the accessible hydroxyl groups of the BC are acetylated, optionally, wherein 1 to 80% of the accessible hydroxyl groups are acetylated, such as 5 to 80%, such as 10 to 80%, such as 20 to 80%, such as 30 to 80%, such as 40 to 80%, such as 50 to 80%, such as 60 to 80%, such as 70 to 80%.

12. The BC suspension according to any one of the preceding claims, which when applied to coat a paper of a grammage of 60 g/m² at a dry coating thickness of 40 μm results in a water vapor transmission rate (WVTR) range of 50-200 g/m²/day at 50% relative humidity, such as 75-175 g/m²/day at 50% relative humidity, such as 85-160 g/m²/day at 50% relative humidity, such as 90-155 g/m²/day at 50% relative humidity, such as 90.8-154.6 g/m²/day at 50% relative humidity.

13. The BC suspension according to any one of the preceding claims, which when applied to coat a paper of a grammage of 60 g/m² at a dry coating thickness of 40 μm results in an oxygen transfer rate (OTR) range of 2.5-40 ml/m²/day/atm, such as 5-35 ml/m²/day/atm, such as 7.5-30 ml/m²/day/atm, such as 10-26 ml/m²/day/atm, such as 11.2-24.2 ml/m²/day/atm.

14. A method for producing a bacterial cellulose (BC) suspension comprising the steps of:
a) Incubating at least one cellulose producing bacteria in a culture medium, and optionally, at least one other microorganism which does not produce cellulose, thereby obtaining BC in said culture medium;
b) Recovering said BC;
c) Optionally, contacting said BC with a functionalization agent, wherein said functionalization agent is selected from nitric acid and acetic anhydride, thereby functionalizing said BC;
d) Converting the recovered BC of step b) and/or the functionalized BC of step c) into a BC suspension comprising 1.6 to 2.3% (w/v) BC solids, said BC suspension comprising BC fiber clusters having a size between 10 nm and 1500 μm, and/or said BC suspension having a viscosity between 1 and 500 Pa·s at a shear stress of 1/s.

15. The method according to claim 14, wherein the BC suspension comprises between 1.6 and 2.2% (w/v) of BC solids, such as between 1.6 and 2.1% (w/v) of BC solids, such as between 1.6 and 2.0% (w/v) of BC solids, such as between 1.6 and 1.9% (w/v) of BC solids, such as between 1.6 and 1.8% (w/v) of BC solids, such as between 1.6 and 1.7% (w/v) of BC solids, such as between 1.7 and 2.3% (w/v) of BC solids, such as 1.7 and 2.2% (w/v) of BC solids, such as between 1.7 and 2.1% (w/v) of BC solids, such as between 1.7 and 2.0% (w/v) of BC solids, such as between 1.7 and 1.9% (w/v) of BC solids, such as between 1.7 and 1.8% (w/v) of BC solids, such as between 1.8 and 2.3% (w/v) of BC solids, such as between 1.8 and 2.2% (w/v) of BC solids, such as between 1.8 and 2.1% (w/v) of BC solids, such as between 1.8 and 2.0% (w/v) of BC solids, such as between 1.8 and 1.9% (w/v) of BC solids, such as between 1.9 and 2.3% (w/v) of BC solids, such as between 1.9 and 2.2% (w/v) of BC solids, such as between 1.9 and 2.1% (w/v) of BC solids, such as between 1.9 and 2.0% (w/v) of BC solids.

16. The method according to any one of claims 14 to 15, further comprising further incubating at least one microorganism, such as a bacteria, producing or being capable of producing at least one cellulytic agent in said culture medium.

17. The method according to any one of claims 14 to 16, wherein said at least one other microorganism which does not produce cellulose and said at least one microorganism producing or being capable of producing at least one cellulytic agent are identical or different.

18. The method according to any one of claims 14 to 17, wherein said at least one cellulose producing bacteria and said at least one microorganism producing or being capable of producing at least one cellulytic agent are identical or different.

19. The method according to any one of claims 14 to 18, wherein the BC has a crystallinity index (CI) of 0.05 to 8, for example between 0.1 and 7, such as between 0.5 and 6.5, such as between 1 and 6, such as 2.5, such as 3, such as 3.5, such as 4, such as 4.5, such as 5, such as 5.5, such as 6, or between 1 and 5, between 2 and 5, between 2 and 4, between 3 and 5, between 3 and 4, between 1 and 2, between 1 and 4, between 1 and 3, or between 2 and 3.

20. The method according to any one of claims 14 to 19, comprising step c), wherein the functionalization agent is nitric acid, whereby at least some of the accessible hydroxyl groups of the BC are substituted with nitro groups, preferably wherein the concentration of nitric acid is 0.3 to 10% (v/v), such as 1 to 10%, such as 1 to 5%, such as 2 to 6%, such as 3 to 8%.

21. The method according to any one of claims 14 to 20, comprising step c) wherein the functionalization agent is acetic anhydride, whereby at least some of the accessible hydroxyl groups of the BC are acetylated, preferably wherein the concentration of acetic anhydride is 1 to 25%, such as 5 to 15%, such as 1 to 10%, such as 20 to 25%.

22. The method according to any one of claims 14 to 21, wherein the functionalization increases the hydrophobicity of the BC, wherein the hydrophobicity is defined as the water contact angle of the BC, optionally, wherein the water contact angle of the BC is increased from 45° to 150°, such as to 50°, such as to 60°, such as to 70°, such as to 80°, such as to 90°, such as to 100°, such as to 110°, such as to 120°, such as to 130°, such as to 140°, such as 145°, such as 150°.

23. The method according to any one of claims 14 to 22, wherein the at least one cellulose producing bacteria is Komagataeibacter rhaeticus and/or Gluconobacter oxydans.

24. The method according to any one of claims 14 to 23, wherein the culture medium comprises a carbon source, selected from glycerol, fructose, sucrose and/or glucose.

25. The method according to any one of claims 14 to 24, wherein the culture medium comprises sucrose at a concentration of 1 to 30%, such as 2 to 10%, such as 2 to 5%, such as 1 to 8%; or such as 4 to 10%, or such as 8 to 15%, or such as 10 to 25%, or such as 15 to 20%.

26. The method according to any one of claims 14 to 25, wherein the culture medium comprises glucose at a concentration of 1 to 30%, such as 2 to 10%, such as 2 to 5%, such as 1 to 8%; or such as 4 to 10%, or such as 8 to 15%, or such as 10 to 25%, or such as 15 to 20%.

27. The method according to any one of claims 14 to 26, wherein the at least one cellulose producing bacteria is Komagataeibacter rhaeticus and/or Gluconobacter oxydans, and optionally wherein the at least one microorganism which does not produce cellulose is Hanseniaspora uvarum, Brettanomyces bruxellensis, Zygosaccharomyces bailii and/or Kloeckera lindneri.

28. The method according to any one of claims 14 to 27, wherein step a) is performed at a temperature between 15 and 30° C., such as between 18 and 28° C., such as between 20 and 26° C., such as between 22 and 24° C.

29. The method according to any one of claims 14 to 28, wherein step a) is performed at room temperature.

30. The method according to any one of claims 14 to 29, wherein the cultivation in step a) is an aerobic fermentation, such as a static aerobic fermentation, such as without shaking.

31. The method according to any one of claims 14 to 30, wherein the cultivation in step a) is an agitated fermentation.

32. The method according to any one of claims 14 to 31, wherein the cultivation in step a) is performed for a period of 2 to 10 days, such as between 2 and 9 days, such as between 3 and 8 days, such as between 4 and 7 days, such as between 5 and 6 days.

33. The method according to any one of claims 14 to 32, wherein the BC produced in step a) is in the form of a BC pellicle.

34. The method according to claim 33, further comprising a step of recovering the BC, preferably by removing it from the surface of the culturing medium or from the interface between surface and culturing medium.

35. The method according to any one of claims 14 to 34, wherein the BC pellicle has a thickness between 0.2 and 2 cm, such as between 0.2 and 1.8 cm, such as between 0.4 and 1.6 cm, such as between 0.6 and 1.4 cm, such as between 0.8 and 1.2 cm, such as about 1.0 cm.

36. The method according to any one of the claims 14 to 35, further comprising a step of sterilisation, preferably said step is performed before steps a-d, most preferably said step is performed before step c.

37. The method according to claim 36, wherein said sterilisation is performed by heat treatment, chemical treatment, enzymatic treatment, irradiation, physical treatment, pressure treatment such as high pressure treatment, and/or filtration.

38. The method according to claim 36, wherein said sterilisation is chemical sterilisation, preferably said chemical sterilisation comprises lowering the pH of a sample, suspension, BC suspension, culture medium, recovered BC, functionalized BC, and/or BC pellicle below pH 7.

39. The method according to claims 36 and/or 38, wherein said sterilisation comprises enzymatic treatment of said sample, suspension and/or culture medium, such as by addition of an enzyme.

40. The method according to any one of claims 36 to 39, wherein the step of sterilisation comprises contacting the BC with a base, such as NaOH and/or NaClO₄, preferably after step b).

41. The method according to any one of claims 36 to 40, wherein the step of sterilisation comprises contacting the BC with a base, preferably after recovering the BC, such as 0.3 to 10% (w/w) NaOH, such as with 0.5 to 9%, such as 0.75 to 8%, such as 1 to 7%, such as 2 to 6%, such as 3 to 5% NaOH.

42. The method according to any one of claims 14 to 41, wherein the BC suspension is prepared by comminution of the recovered and/or the functionalized BC.

43. The method according to any one of claims 14 to 42, wherein the recovered and/or the functionalized BC is comminuted using at least one of the comminution methods and/or instruments selected from the group consisting of mechanical shearing stress, wet grinding mill, cutting, crushing, trituration, ultrasonication, aqueous counter-collision, high-pressure homogenization, microfluidizer, comminution using an IKA Turrax, comminution using a Silverson homogenizer, and comminution using a supermasscolloider, such as Masuko grinding stone, preferably comminution using a supermasscolloider, such as Masuko grinding stone.

44. The method according to any one of claims 42 to 43, wherein the comminution comprises grinding the recovered BC, such as grinding with a supermasscolloider, such as a Masuko grinding stone, preferably at a speed of 500 to 3000 rpm, between 600 and 2800 rpm, such as between 700 and 2600 rpm, such as between 800 and 2400 rpm, such as between 900 and 2200 rpm, such as between 1000 and 2000 rpm, such as between 1200 and 1800 rpm, such as between 1400 and 1600 rpm.

45. The method according to any one of claims 42 to 44, further comprising a step of centrifugating the BC before and/or after the comminution.

46. The method according to any one of claims 42 to 45, further comprising a step of heating the BC after and/or during the comminution, optionally wherein the heating is performed at a temperature between 30° C. and 200° C., such as between 30° C. and 200° C., such as between 40° C. and 190° C., such as between 50° C. and 180° C., such as between 60° C. and 170° C., such as between 70° C. and 160° C., such as between 80° C. and 150° C., such as between 90° C. and 140° C., such as between 100° C. and 130° C., such as between 110° C. and 120° C., and/or wherein the heating is performed for 5 min to 10 h, such as 10 min to 9 h, such as 20 min to 8 h, such as 30 min to 7 h, such as 1 h to 6 h, such as 2 h to 5 h, such as 3 h to 4 h.

47. The method according to any one of claims 14 to 46, further comprising a step of reacting and/or mixing said BC suspension with an additional component, such as glycerol, polyvinyl alcohol (PVOH), pectin, casein, starch, modified and/or unmodified silicon dioxide, and/or titanium oxide.

48. Use of the BC suspension as defined in any one of the preceding claims for coating a material, such as the surface of a material.

49. A method for coating a material, said method comprising the method from any one of claims 14 to 47, thereby obtaining a BC suspension as defined in any one of the preceding claims, said method further comprising a step of coating a material, such as the surface of a material, with the BC suspension, thereby obtaining a coated material.

50. The use or the method according to any one of claims 48-49, wherein the material is selected from the group consisting of paper, paperboard/cardboard, foodstuff, cosmetics, and textiles, such as woven and/or non-woven textiles, preferably the material is specialty paper, corrugated fibreboard paper, cellulose- and/or fiber-based materials.

51. The use or the method according to any one of claims 48-50, wherein the BC suspension is applied with a coating thickness between 1 and 40 μm, such as between 1 and 10 μm, such as 1 μm, such as 2 μm, such as 3 μm, such as 4 μm, such as between 5 and 10 μm, such as 5 μm, such as 6 μm, such as 7 μm, such as 8 μm, such as 9 μm, such as 10 μm, such as between 10 and 20 μm, such as 12 μm, such as 14 μm, such as 16 μm, such as 18 μm, such as 20 μm, such as between 20 and 40 μm, such as between 20 and 30 μm, such as 22 μm, such as 24 μm, such as 26 μm, such as 28 μm, such as 29 μm, such as between 30 and 40 μm, preferably the coating thickness is at the most 10 μm such as less than 10 μm.

52. The use or the method according to any one of claims 48-51, wherein the BC suspension is applied with a wet coating thickness between 10 and 150 μm, such as between 25 and 125 μm, such as between 30 and 100 μm, such as 10 μm, such as 20 μm, such as 30 μm, such as 40 μm, such as 50 μm, such as 60 μm, such as 70 μm, such as 80 μm, such as 100 μm, such as 110 μm, such as 120 μm, such as 130 μm, such as 140 μm, such as 150 μm, such as between 10 and 50 μm, such as between 30 and 75 μm, such as between 50 and 110 μm, such as between 75 and 150 μm, such as between 90 and 100 μm, preferably the wet coating thickness is 100 μm.

53. The use or the method according to any one of claims 48-52, wherein coating paper or cardboard with the BC suspension generates a coated paper or cardboard with a grammage between 25 and 500 g/m², such as between 40 and 450 g/m², such as between 40 and 400 g/m², such as between 50 and 400 g/m², such as between 50 and 375 g/m², such as between 50 and 360 g/m².

54. The use or the method according to any one of claims 48-53, wherein the coated material is a coated paper or cardboard with a porosity between 1 and 250 mL/min, such as between 1 and 150 mL/min, such as between 2.5 and 175 mL/min, such as between 2.5 and 150 mL/min, such as between 5 and 250 mL/min, such as between 5 and 175 mL/min, such as between 5 and 150 mL/min.

55. The use or the method according to any one of claims 48-54, wherein the coated material has a water vapor transmission rate (WVTR) less than 75 g/m²/day, such as less than 60 g/m²/day, such as less than 50 g/m²/day, such as less than 45 g/m²/day, such as less than 40 g/m²/day, such as less than 30 g/m²/day, or less.

56. The use or the method according to any one of claims 48-55, wherein with the coated material has a WVTR that is two to six times lower than the WVTR of the material before the coating, such as two times, such as three times, such as four times, such as five times, such as six times lower compared to the WVTR of the material before the coating.

57. The use or the method according to any one of claims 48-56, wherein the coated material has an oil absorption less than 10 g/m², such as less than 9.5 g/m², such as less than 9 g/m², such as less than 8.5 g/m², such as less than 8 g/m², such as less than 7.5 g/m², such as less than 7 g/m², or less.

58. The use or the method according to any one of claims 48-57, wherein the coated material has an oil absorption that is two to six times lower than the oil absorption of the material before the coating, such as two times, such as three times, such as four times, such as five times, such as six times lower compared to the oil absorption of the material before the coating.

59. The use or the method according to any one of claims 48-58, wherein the coated material has a grease resistance of a KIT number of at least 6, such as at least 7, such as at least 8, such as at least 9, such as at least 10, such as at least 11, such as 12.

60. The use or the method according to any one of claims 48-59, wherein the coated material has a grease resistance that is two to six KIT points/numbers higher than the grease resistance of the material before the coating when said grease resistance is measured or tested using the KIT test, such as two KIT points/numbers, such as three KIT points/numbers, such as four KIT points/numbers, such as five KIT points/numbers, such as six KIT points/numbers higher compared to the grease resistance of the material before the coating.

61. The use or the method according to any one of claims 48-60, wherein the material is coated with the BC suspension after being coated with another agent, optionally, wherein the material is coated with another agent after being coated with the BC suspension.

62. The use or the method according to any one of claims 48-60, wherein the BC suspension further comprises an additional component, such as glycerol, polyvinyl alcohol (PVOH), pectin, casein, starch, modified and/or unmodified silicon dioxide, and/or titanium oxide.

63. The use or the method according to claim 61-62, wherein the other agent and/or additional component is polyvinyl alcohol (PVOH), nitrocellulose, modified and/or un-modified silicon dioxide.

64. The use or the method according to any one of claims 61-63, wherein the modified silicon oxide is silicon dioxide (SiO₂) modified with dimethylsylilates particles having a sylilate tail length of H8 to H22, such as a sylilate tail length of H10 to H20, such as a sylilate tail length of H15 to H20, such as sylilate tail length of H15 to H18, such as a sylilate tail length of H15, such as a sylilate tail length of H16, such as a sylilate tail length of H17, such as a sylilate tail length of H18, optionally the modified SiO₂ is selected from SiO₂ modified with dimethylsylilates H15 particles with BET surface of 130-170 m²/g and SiO₂ modified with dimethylsylilates H18 particles with BET surface of 170-230 m²/g.

65. Use of the BC suspension according to any one of claims 1 to 13 or as defined in any one of claims 14 to 64 as particle stabilizer (particle stabilizator) and/or emulsion stabilizer (emulsifier) and/or thickener.

66. A method of producing a particle stabilizer (particle stabilizator) and/or emulsion stabilizer (emulsifier) and/or thickener comprising the BC suspension according to any one of claims 1 to 13 or as defined in any one of claims 14 to 64, said method comprising performing the method according to any one of claims 14 to 64 to obtain the BC suspension.
